(19) United States
(12) Patent Application Publication
(10) Pub. No.: US 2006/0166189 A1
(43) Pub. Date: Jul. 27, 2006

(54) Methods for detecting invasion of a cell

(76) Inventor: Hana Golding, Rockville, MD (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/536,860

(22) PCT Filed: Nov. 24, 2003
(86) PCT No.: PCT/US03/37677

§ 371 Date: Jan. 6, 2006

Related U.S. Application Data
(60) Provisional application No. 60/429,767, filed on Nov. 27, 2002.

(51) Int. Cl. C12Q 1/70

(52) U.S. Cl. 435/5

(57) ABSTRACT

The present invention provides a rapid method to determine invasion of a cell by an invasion.

### CROSS-REFERENCE TO RELATED APPLICATIONS

This application claims priority to U.S. Provisional Patent Application No. 60/429,767, filed Nov. 27, 2002, which is herein incorporated by reference in its entirety.

### GOVERNMENT FUNDING

The invention described herein was developed with support from the Department of Health and Human Services. The U.S. Government has certain rights in the invention.

### FIELD OF THE INVENTION

The invention relates generally to methods to determine the presence of an antibody in a specimen. More specifically, the invention includes methods to detect and titer antibodies in a specimen that recognize a pathogen, such as a smallpox virus. In addition, the invention can be used to detect and titer antibodies in a specimen that recognize pathogenic bacteria. Such methods can be used to assess induction of an immunogenic response by a vaccine directed against a pathogen.

### BACKGROUND OF THE INVENTION

Antibody binding to a pathogen that disallows productive infection by the pathogen is called neutralization. Neutralization of viruses and other pathogens by antibodies has been extensively studied during the last century, and many mechanisms for neutralization have been proposed. In regard to viral infection, these mechanisms include aggregation, inhibition of viral entry by inhibition of attachment and inhibition of fusion with the target cell, as well as post entry mechanisms, such as interference with primary and secondary uncoating of the genetic information of the virus. It has been suggested that some antibodies may neutralize a virus by acting via several mechanisms simultaneously or sequentially. It has also been suggested that antibodies generally act by coating the surface of the pathogen, and the neutralized pathogens represent entities on which this coating has reached a critical density. This antibody coat then prevents the pathogen from interacting properly with the target cell, thereby interfering with the initiation to a productive infection. Differential effects of the antibody coat on distinct viruses, such as inhibition of attachment, viral entry, or apparent post entry effects, may be due to differences in the epitope being recognized, but also may in part be explained by differences in virus biology rather than by the direct induction of specific events by the antibody.

Recently, small organic molecules, peptides, and nucleotides have been investigated as anti-pathogenic agents, and in particular, anti-viral agents. Their anti-pathogenic mechanisms are thought to be as varied as those for antibodies.

Consequently, researchers have long sought to develop in vitro assays that can reliably predict the anti-pathogen activity of antibodies and other materials in vivo. These kinds of assays could be used to gain information regarding the mechanisms that antibodies use in vivo to neutralize pathogens.

Such information is important for both vaccine design and for development of agents that can be used in passive antibody administration. In addition, researchers have sought reliable assays that can be used to determine the presence and concentration of antibodies in a specimen. These assays are very important for assessing the ability of a vaccine to elicit an immune response by a vaccine. These assays can also be used to determine whether a patient has been exposed to a pathogen, such as smallpox. Currently, these assays are technically demanding and require extended periods of time to provide needed results. Accordingly, methods that can be used to quickly and reliably determine antibody concentrations, and determine the ability of a vaccine to elicit an immune response in a recipient are needed.

### SUMMARY OF THE INVENTION

The present invention relates to the discovery of methods that allow rapid detection of the invasion of a cell by an invasin, or determination of whether a specimen contains an antibody that decreases invasion of a cell by an invasin. In particular embodiments, the methods can be used to determine if an agent modulates invasion of a cell by an invasin, or determine if a sample contains an antibody that reduces invasion of a cell by an invasin. Additional embodiments of the invention include methods to determine if a human was exposed to smallpox, or determine if a smallpox vaccine can cause production of antibodies in a human. The methods of the invention can also be used to determine if a specimen contains an antibody that binds to a preselected antigen, or determine if an antibody binds to a receptor used by an invasin to invade a cell. Methods to determine if an agent modulates antibody-mediated infection of a cell, determine if an antibody mediates transport of an invasin across a cell monolayer, and assay viral load in vivo are provided. The methods can also be used to diagnose infection of a human by an invasin.

The methods are based on the use of an invasin that encodes a detectable label which allows the invasin to be detected when it invades a cell. Generally, the methods of the invention are conducted by incubating a mixture containing a cell, and an invasin that encodes a detectable label, under conditions wherein the invasin can invade the cell, and then detecting the detectable label in the cell to determine if the invasin invaded the cell. Accordingly, the methods of the invention can be used to determine if a candidate agent modulates invasion of a cell by an invasin, determine if a specimen contains an antibody that decreases invasion of a cell by an invasin, determine if a human has been exposed to smallpox, determine if a smallpox vaccine was able to elicit an immune response in a human, determine if a specimen contains an antibody that binds to a preselected antigen, determine if an antibody binds to a receptor used by an invasin to invade a cell, determine if a candidate agent decreases antibody-mediated infection of a cell by an invasin, and determine if an antibody mediates transport of an invasin across a cell monolayer. The methods of the invention may also be used to assay invasin load in an organism in vivo. The invention also provides kits that contain materials for practicing the methods of the invention.

The invasin may be any type of organism that can encode a detectable label, and invade a cell through action by the cell or action by the invasin. Specific examples of invasins include bacteria or viruses. Preferably, the invasin is a pathogenic bacterium. More preferably, the invasin is a non-enveloped virus. Even more preferably, the invasin is an enveloped virus. Still even more preferably, the enveloped virus is a human immunodeficiency virus. Yet still even more preferably, the enveloped virus is a poxvirus. Even yet still even more preferably, the enveloped virus is smallpox. Most preferably, the enveloped virus is a vaccinia virus.

A candidate agent may interact with a cell to prevent invasion of the cell by an invasin. The interaction will involve any of the mechanisms by which an invasin may invade a cell. These mechanisms include action by the cell or action by the invasin. Examples of such actions include, pinocytosis, phagocytosis, endocytosis, receptor-mediated endocytosis, and Fc-receptor mediated endocytosis, and viral insertion of nucleic acid into a cell. A candidate agent may interact with any of these mechanisms of action. Preferably, the candidate agent interacts with a receptor on the cell to prevent invasion of the cell by an invasin. More preferably, the candidate agent interacts with a cytokine receptor on the cell. Even more preferably, the candidate agent interacts with a chemokine receptor. Still even more preferably, the candidate agent interacts with a CD4 receptor (cluster of differentiation 4 receptor). Yet still even more preferably, the candidate agent interacts with an Fc receptor (fragment crystallizable receptor) on the cell. Most preferably, the candidate agent associates with a heparin sulfate receptor or an epidermal growth factor receptor on the cell.

The candidate agent may alternatively or additionally interact with the invasin to block invasion of a cell. Preferably, the candidate agent prevents fusion of the cellular membrane with the membrane of an enveloped virus. More preferably, the candidate agent interacts with a component of the invasin that is a ligand for a cellular receptor. Even more preferably, the candidate agent interacts with hemaglutinin on the invasin. Still even more preferably, the candidate agent interacts with an HIV gp120 protein. Most preferably, the candidate agent interacts with a glycosaminoglycan or signaling receptor used by a vaccina virus to bind to a heparin sulfate or epidermal growth factor receptor.

The structure of the candidate agent may be a peptide, nucleotide, sugar, lipid, or inorganic or organic compound that operates to prevent cellular invasion by an invasin. Preferably, the candidate agent is a peptide that is bound by a cellular receptor and blocks interaction of the invasin with the cell. More preferably the candidate agent is an enzyme that inactivates a cellular receptor used by the invasin to invade the cell. Even more preferably, the candidate agent is an enzyme that inactivates a ligand on an invasin that is used to invade a cell. Still even more preferably the candidate agent is an antibody that binds to a cellular receptor or a ligand on an invasin. Yet still even more preferably, the candidate agent is an inorganic or organic compound that prevents invasion of the cell by an invasin. Most preferably the candidate agent is a pharmaceutical composition that prevents invasion of a cell by an invasin.

The cell may be any cell that can be invaded by an invasin that encodes a detectable label, and in which the detectable label encoded by the invasin can be expressed. Preferably, the cell is a mammalian cell. More preferably, the cell is a BSC1 cell. Still more preferably, the cell is a Vero cell. Even still more preferably, the cell is a human cell. Most preferably, the cell is a HeLa cell.

A specimen may be any material that is suspected of containing antibodies. Preferably, the specimen is obtained from an animal and includes blood, serum, or tissue. More preferably, the specimen is a processed antibody preparation. Even more preferably, the specimen is a pharmaceutical preparation. Most preferably the specimen is a vaccinia immunoglobulin G (VIG) preparation.

A detectable label that is encoded by the invasin may be any label that can be encoded by an invasin and expressed when the invasin invades a cell, or expressed within the invasin. Preferably, the detectable label is a fluorescent protein, such as green, red, yellow, cayenne, or blue fluorescent protein. More preferably, the detectable label is an enzyme, such as luciferase, peroxidase, alkaline phosphatase, and xanthine oxidase. Most preferably, the detectable label is β-galactosidase.

A preselected antigen may be encoded and expressed by an invasin such that the preselected antigen is displayed on the surface of the invasin. If the invasin is an enveloped virus, the virus may encode the preselected antigen such that the preselected antigen is expressed and displayed on the surface of a packaging cell used to package the enveloped virus. Enveloped viruses that are packaged by such a packaging cell will also display the preselected antigen on the surface of their viral membrane because it is derived from the cell membrane having the predetermined antigen displayed on its surface. A packaging cell may express and display the preselected antigen on the surface of the packaging cell. Enveloped viruses that are packaged by such a packaging cell will also display the preselected antigen on their surface as described previously. Preferably, the preselected antigen is a predetermined peptide. More preferably, the preselected antigen is a fusion protein having a predetermined peptide operably linked to a membrane localization signal. Even more preferably, the predetermined polypeptide is a polypeptide that is used as a subunit vaccine. Most preferably, the preselected polypeptide is an antigenic portion of the HIV gp120 protein.

A cell membrane preparation obtained from nearly any cell that can be invaded by an invasin may be used to raise monoclonal antibodies against components of the cell membrane. Preferably, a monoclonal antibody is raised against a cell membrane preparation from a cell that occurs in nature and that is invaded by an invasin. More preferably, the cell is transfected with a nucleic acid segment that encodes a receptor used by an invasin to invade the cell. Most preferably, the wild-type cell is not invaded by an invasin prior to being transfected with a nucleic acid segment that encodes a receptor used by an invasin to invade the cell which allows the invasin to invade the transformed cell. Preferably the monoclonal antibody was raised against a mammalian cell membrane preparation. More preferably the monoclonal antibody was raised against a human cell membrane preparation.

Nearly any cell that displays an Fc receptor (fragment crystallizable receptor) that is used by an invasin to invade the cell through antibody-mediated invasion may be used to determine if a candidate agent modulates antibody-mediated invasion of the cell by the invasin. Preferably, the cell that displays the Fc receptor is a wild-type cell. More preferably, the cell is a recombinant cell that was transfected with a nucleic acid segment encoding an Fc receptor that enables the invasin to invade the cell by antibody-mediated invasion. Most preferably, the cell does not display an Fc receptor used by an invasin to invade the cell prior to being transfected with a nucleic acid segment that encodes such an Fc receptor.

Nearly any cell that forms tight junctions and a monolayer may be used within the methods of the invention that involve transport of an invasin across a cell monolayer. Preferably, the cells of the monolayer are mammalian cells. More preferably, the cells of the cell monolayer are human cells. Most preferably, the cells of the cell monolayer are human intestinal cells.

The invention provides methods to assay invasin load in vivo. Generally, the methods involve infecting an organism with an invasin that encodes a detectable label, obtaining specimens from the organism, and detecting the detectable label in the specimens. Accordingly, these methods allow the spread of the invasin and the quantity of invasin throughout the animal to be determined. Preferably, the organism is an avian. More preferably, the organism is a mammal such as a dog, cat, rabbit, rat, or mouse. Preferably, the organism has a functioning immune system. More preferably the organism is immunodeficient. Preferably, the specimen is a tissue, such as liver, spleen, lymph node, or ovary. More preferably, the specimen is blood. Most preferably, the specimen is serum. The method may include quantifying the amount of invasin in the specimen. Preferably the method includes determining the kinetics of viral dissemination. The method may include determining if administration of an antiinvasin modulates invasin dissemination. Preferably the antiinvasin is a vaccine, an antibody preparation, a monoclonal antibody, a polyclonal antibody, an enzyme, a peptide, a compound, a pharmaceutical composition, or any combination thereof. The antiinvasive may be administered before the organism is infected with the invasin. The antiinvasive may be administered after the organism is infected with the invasin.

The invention provides a kit containing packaging material and an invasin that encodes a detectable label. The invention also provides a kit containing packaging material, an invasin that encodes a detectable label, and at least one cell that can be infected by the invasin.

### BRIEF DESCRIPTION OF THE DRAWINGS

FIG. 1A illustrates a β-Galactosidase enzymatic standard curve.

FIG. 1B illustrates a Vaccinia neutralization curve.

FIG. 2A illustrates VIG control charts showing the neutralizing antibody 50% inhibition values in μg/ml (ID(50)) for repeated tests of VIGIV resulting from 32 tests conducted on MBL VIGRV lot #2. Horizontal lines depict the specimen mean and the 95% upper and lower control limits (UCL and LCL respectively).

FIG. 2B illustrates VIG control charts showing the neutralizing antibody 50% inhibition values in μg/ml (ID(50)) for repeated tests of VIGIV resulting from 11 tests conducted on FDA Standard (MBL VIGIV lot #1). Horizontal lines depict the specimen mean and the 95% upper and lower control limits (UCL and LCL respectively).

FIG. 2C illustrates VIG control charts showing the neutralizing antibody 50% inhibition values in μg/ml (ID(50)) for repeated tests of VIG resulting from 17 experiments conducted with Baxter VIG. Horizontal lines depict the specimen mean and the 95% upper and lower control limits (UCL and LCL respectively).

### DETAILED DESCRIPTION OF THE INVENTION

With the renewed threat of smallpox as a bioterrorism agent (Henderson, Science, 283:1279 (1999); Henderson, Emerg. Infect. Dis., 5:537 (1999); Henderson and Fenner, Clin. Infect. Dis., 33:1057 (2002); Henderson et al., JAMA, 281:2127 (1999); Lane et al., N. Eng. Jour. Med., 281:1201 (1969)), major efforts are underway in the following areas: a) development of new stocks of smallpox vaccines in new cell substrates (diploid cell lines, Vero cells), b) Development of more attenuated strains of vaccinia as vaccine candidates for immunodeficient individuals, and c) production of high titer vaccinia IgG (VIG) that will be required to treat individuals with severe vaccinia-associated adverse reactions (Goldstein et al., Pediatrics, 55:342 (1975)) and for prophylactic usage in high risk individuals and pregnant women (Katz, J. Biol. Stand., 15:389 (1987)). In parallel, it is essential to develop laboratory-based assays to evaluate the potency of the new vaccines and VIG products. One of the key assays to assess humoral immune responses in vaccinees and the potency of VIG preparations is a vaccinia neutralization assay. The current neutralization assays are all based on plaque reduction neutralizing titers (PRNT). They are slow (4-7 days), require relatively large volume of test articles (since they are conducted in 6-well or 12-well plates), they are also somewhat subjective and operator-dependent since the read-out is visual (Mack et al., Am J. Trop. Med. Hyg., 21:214 (1972); Paoletti, Proc. Natl. Acad. Sci., 93:11349 (1996).

Therefore, the PRNT assays are considered difficult to standardize and validate, and to transfer between laboratories. Furthermore, due to the low throughput, only limited numbers of specimens can be tested in each assay set up, making it impractical for testing of hundreds of specimens from clinical trials.

The current invention provides novel methods to measure the invasion of a cell by an invasin. For example, the methods of the invention may be used to assay vaccinia neutralization. The methods are generally based on the expression of a detectable label. An example of a detectable label is the bacterial β-galactosidase enzyme (β-Gal). The gene encoding the bacterial β-galactosidase enzyme may be put under the control of a synthetic E/L promoter (Chakrabarti et al., Biotechniques, 23:1094 (1997)). It is demonstrated herein that the new assay is rapid (24 hr), of equal sensitivity to PRNT assays, reproducible, objective, and easy to transfer. In addition, results with a second reporter gene assay based on a vaccinia-EGFP recombinant virus are disclosed herein. The expression of the GFP may be induced by IPTG (Bleckwenn, Biotechnol. Prog., 19:130 (2003)). The two assays provide high throughput capabilities and may be established in clinical laboratories for the evaluation of multiple specimens from clinical trials.

The novel methods described herein may be used to replace the traditional PRNT assays. For example, it has been demonstrated that the β-Gal reporter gene assay, using a recombinant vaccinia virus vSC56, is rapid (24 hr), sensitive, reproducible, and produces very similar results to those obtained in two different PRNT assays. Since the readout is an enzymatic reaction resulting in a substrate color change, it can be read by an ELISA reader instrument currently available in most clinical laboratories. This readout is objective and can be enhanced by the inclusion of a β-galactosidase standard curve in each assay. The new methods are easy to validate and transfer to other laboratories. The new methods also allow freezing of infected cells prior to conducting the enzymatic reaction. This property of the assay will provide significant advantage to laboratories that chose to set up several groups of experimental sera and then run all the enzymatic reactions in one batch. The methods of the invention also allow investigators to repeat determinations on individual specimens to confirm their results. The new methods are sensitive enough to detect both high-titer and low-titer antibodies in sera that may be neutralizing for an invasin. Accordingly, the methods of the invention may be used to screen clinical specimens and preparations of IVIG as a back-up for VIG in case of shortage induced by a mass vaccination campaign. Such a shortage could occur in the event that a massive smallpox vaccination campaign became necessary.

The methods of the invention may be valuable for large-scale clinical trials of new vaccines, such as smallpox vaccines, produced in diploid cell substrates and Vero cells. The methods of the invention may also be used in additional trials with more attenuated vaccinia strains, such as modified vaccinia Ankara (MVA) (Frey et al., N. Eng. J. Med. 346:1275 (2002); Ramirez et al., J. Virol. 74:923 (2000); Meyer et al., J. Gen. Virol., 72:1031 (1991)), which are defective in virion maturation in mammalian cells. Such strains could be much safer for vaccination of individuals with known immunodeficiencies.

In order to further establish the in vivo biological correlation of the ill vitro neutralization assays of the invention, a mouse lethality model using SCID mice has been established. The difference in neutralization titer observed in the β-Gal assay correlated positively with significant difference in the protective efficiency against lethal infection of SCID mice with vaccinia (Wyeth). The most likely explanation for the lower protective activity of VIG (16.5%) compared with the VIGIV (5%) is the fact that it was produced 8 years ago and passed its expiration date.

Recombinant invasions, such as vaccinia viruses, expressing other reporter genes such as green fluorescent protein (GFP) and luciferase (LUC) (Kempe et al., Pediatrics, 42:980 (1968); Frey et al., N. Eng. Jour. Med., 346:1275 (2002)) may also be used in the methods of the invention. Preliminary studies with an inducible EGFP-vaccinia recombinant demonstrated good sensitivity at MOI of 0.05 pfu/cell in a 48 hr neutralization assay. The ID₅₀ values obtained for FDA Standard and several other VIG preparations were in good agreement with those obtained in the β-Gal assay (Table 6). Accordingly, the methods of the invention may be used in high-throughput methods.

I. Definitions

Abbreviations: vaccinia immune globulin (VIG); plaque reduction neutralizing titers (PRNT); β-galactosidase (β-Gal); green fluorescent protein (GFP); isopropyl-β-D-thiogalactopyranoside (IPTG); enzyme linked immunosorbant assay (ELISA); luciferase (LUC); multiplicity of infection (MOI); plaque forming units (PFU); inhibitory dilution (ID); peripheral blood mononuclear cell (PBL).

The term “altered antibody” includes antibody fragments that include, but are not limited to, Fab fragments, single-chain antibodies, antibodies having altered amino acid linkages, cross-linked antibodies, conjugated antibodies, humanized antibodies, and the like.

The term “antibody-mediated infection” refers to enhanced invasion of a cell by an invasin due to antibody binding. Such enhanced invasion of a cell by an antibody-invasin complex is well known in the art. For example, for HIV-1, both Fc receptor-mediated and complement-mediated antibody-dependent enhancement of infection have been described. In another example, Fc receptor-mediated antibody-dependent enhancement of cell invasion is known for dengue virus, West Nile virus, influenza A viruses, HIV-1, HIV-2, and Epstein-Barr virus. Fields Virology, 4^{th} edition, eds. David Knipe and Peter Howley, Lippincott Williams & Williams, Philadelphia, Pa. 2001.

The term “antibody preparation” refers to a formulation containing purified antibodies. For example, an antibody preparation includes, but is not limited to, pharmaceutical formulations containing antibodies. Such pharmaceutical formulations can be administered to patients that are immune compromised, such as the elderly, transplant patients, and HIV positive persons. An example of an antibody preparation is vaccinia immunoglobulin G (VIG). Such antibody preparations can be prepared from antibodies obtained from serum, antibodies obtained from the supernatant of antibody secreting cells, antibodies obtained from tissue, and the like. The antibody preparations may be prepared from monoclonal and polyclonal antibodies. The antibody preparations can also contain ingredients in addition to antibodies. Such ingredients may be salts, isotonic solutions, buffers, injectable formulations, and the like.

The term “interacts” refers to the interaction of an agent with an invasin or a cell. The interaction may be a physical interaction, such as ligand binding by a receptor, antibody binding to a receptor, and the like. The interaction may also be a modification of a receptor or a ligand by an agent. For example, the agent may modify a cell or an invasin by phosphorylation, glycoslylation, dephosphorylation, deglycosylation, proteolytic cleavage, and the like.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance. Such cloning vectors are well known in the art and are available commercially from a large number of sources.

The term “compound” refers to a chemical structure that is intended for use in preventing invasion of a cell by an invasin, or replication of the invasin within a cell. Examples of numerous compounds are described in the official United States Pharmacopeia, official National Formulary, or any supplement to them. Briefly, examples of compounds include, hydrocarbons, interferons, cyclic organic molecules, bicyclic organic molecules, aryl organic molecules, alkyl organic molecules, and the like. Specific examples of compounds known to be useful for treating viral infections include, idoxuridine, vidarabine, trifluridine, acyclovir, famciclovir, penciclovir, valacyclovir, ganciclovir, foscamet, ribavirin, amantadine, rimantadine, and cidofovir. Specific examples of compounds useful for treating microbial infections include, β-lactam antibiotics, cephalosporins, aminoglycosides, macrolides, tetracyclines, quinolones, and sulfonamides. Numerous compounds having antiinvasin activity are known and have been described. Merck Manual, Merck Research Laboratories, Whitehouse Station, N.J. 17^{th} edition, eds, Beers and Berkow 1999; Merck Index, Merck Research Laboratories, Whitehouse Station, N.J., 13^{th} ed., 2001. It is envisioned that numerous compounds that are known antiinvasives and modifications thereof can be identified according to, and used within, the methods of the invention.

The term “control elements” refers to nucleic acid sequences that are operably linked to a second nucleic acid sequence and control expression of the second nucleic acid sequence. Examples of control elements include promoters, terminators, poly A sites, ribosome binding sites, and the like.

The term “detectable label” refers to a marker that is expressed by a cell and that may be detected. Examples of such detectable labels include, but are not limited to, fluorescent proteins and enzymes. Enzymes that are expressed as detectable labels often will convert a substrate into an easily detected form. Numerous examples of such enzymes are known in the art. Enzymes include β-galactosidase, chloramphenicol transferase, peroxidase, and the like. Sambrook et al., Molecular Cloning: A Laboratory Manual, 3rd edition, Cold Spring Harbor Press, Cold Spring Harbor, N.Y. (2001); Harlow et al., Antibodies: A Laboratory Manual, page 319 (Cold Spring Harbor Pub. 1988).

The term “endogenous” refers to a nucleic acid sequence or segment that is associated with a wild-type organism without having been artificially transferred to the wild-type organism.

The term “exogenous” refers to a nucleic acid sequence or segment that is not associated with a wild-type organism and has been introduced into the organism through artificial means, such as through transformation.

The term “enveloped virus” refers to a virus that forms a viral particle that is surrounded with a membrane. The membrane of such a virus is often derived from the cell in which the virus replicates. The membrane may be decorated with membrane associated components normally displayed on the surface of the cell from which the membrane was derived. For example, the membrane may be decorated with receptors, carbohydrates, cell adhesion molecules, and the like that are derived from the cell. Enveloped viruses may be constructed to display specific components on the surface of their envelopes by expressing the specific component in the cell in which the enveloped virus is packaged. For example, a virus can be made to display a peptide used to manufacture a vaccine on the surface of its envelope by expressing a fusion protein having the peptide fused to a cell surface localization signal within the cell in which the virus is packaged. Alternatively, a recombinant enveloped virus can be constructed to express a fusion protein having the peptide fused to a cell surface localization signal such that a cell infected with the recombinant virus will express the fusion protein on its surface. The envelope of a virus replicated within the cell will include the fusion protein. Examples of enveloped viruses include, but are not limited to, vaccinia, herpesvirus, orthomyxovirus, rhabdovirus, alphavirus, retrovirus, and influenza viruses. Many enveloped viruses are known in the art. Fields Virology, 4^{th} edition, eds. David Knipe and Peter Howley, Lippincott Williams & Williams, Philadelphia, Pa. 2001; Flint et al., Principles of Virology, ASM Press, Washington, D.C. 2000.

The term “fluorescent protein” refers to a protein that is fluorescent and that may be used as a detectable label. Many fluorescent proteins are known. Examples of such proteins include, but are not limited to, numerous red fluorescent proteins, yellow fluorescent protein, and green fluorescent proteins. Many fluorescent proteins are commercially available. BD Biosciences, Clontech, 1020 East Meadow Circle, Palo Alto, Calif. 94303.

The term “immunogenic specimen” refers to a biological specimen obtained from a human that contains antibodies produced by the human against an antigen. Examples of immunogenic specimens include, but are not limited to, blood, fractionated blood, serum, fractionated serum, lymphoid tissue, and purified forms of blood, serum, and lymphoid tissues.

The term “immunoseparate” refers to the use of an antibody to isolate an epitope containing component to which the antibody binds. Numerous methods may be used to immunoseparate an epitope containing component. For example, the antibody may be immobilized on a support and the epitope containing component may then be contacted with the immobilized antibody. The component may then be eluted from the support through use of methods known in the art, such as washing with a buffer having a high salt concentration. In another example, the epitope containing component may be immunoprecipitated with an antibody. Magnetism may be used to immunoseparate the epitope containing component by conjugating an antibody to a metal bead or a magnetic bead. Monoclonal and polyclonal antibodies may be used for immunoseparation.

The term “invade” or “invasion” includes infect, phagocytosis, endocytosis, includes intracellular replication, includes internalization within a cell without replication of invasin.

The term “invasin” refers to a biological entity that can invade a cell through the action of the biological entity, through action of the cell, or through action of the biological entity and action of the cell. An invasin can be pathogenic or non-pathogenic. Examples of invasions include, but are not limited to, bacteria and viruses. Allen et al., Inf. Immun., 69:4673 (2001); Klasse and Saffentau, J. Gen. Virol., 83:2091 (2002).

The term “labeled invasin” includes biological materials that can invade a cell and express a detectable label. Examples of biological materials include, but are not limited to, bacteria, viruses, and the like. Such biological materials include pathogens and non-pathogens. Entry of an invasin into a cell may proceed through action of the cell or through action of the invasin. For example, an invasin may passively enter a cell through pinocytosis, phagocytosis, endocytosis, receptor-mediated endocytosis, and the like. An invasin may also enter the cell through action of the invasin, such as the entry of a cell by a virus. The detectable label may be expressed by the invasin or expressed within the cell upon entry of the invasin into the cell. For example, a bacteria may encode a detectable label that is expressed when the bacteria is inside or outside of a cell. In another example, a virus may encode a detectable label that is expressed after the virus has entered a cell. The detectable label may be encoded by an exogenous DNA segment that is introduced into an invasin through recombinant techniques that are well known in the art. Sambrook et al., Molecular Cloning: A Laboratory Manual, 3rd edition, Cold Spring Harbor Press, Cold Spring Harbor, N.Y. (2001).

The term “ligand” refers to any molecule that is recognized by a receptor. Numerous examples of ligands are known and include, but are not limited to, gp120 of HIV, HA of influenza, and the like. Fields Virology, 4^{th} edition, page 89, eds. David Knipe and Peter Howley, Lippincott Williams & Williams, Philadelphia, Pa. 2001. Flint et al., Principles of Virology, ASM Press, Washington, D.C. 2000.

The term “modulates” refers to an increase or decrease in a detectable characteristic.

The term “non-enveloped virus” refers to a virus that forms a viral particle that is not surrounded by an envelope. Such viral particles often display an outer protein coat formed by assembly of capsid proteins. An example of a non-enveloped virus includes, but is not limited to, adenovirus. Many non-enveloped viruses are known in the art. Fields Virology, 4^{th} edition, eds. David Knipe and Peter Howley, Lippincott Williams & Williams, Philadelphia, Pa. 2001.

The term “operably linked” means joined as part of the same nucleic acid molecule. For example, DNA operably linked to a promoter is “under transcriptional initiation regulation” of the promoter.

The term “pathogenic bacterium” refers to a bacterium that is a causative agent in disease. Examples of pathogenic bacteria include, Shigella, Listeria, Salmonella, Tuberculosis, B. abortis, Chlamydia, Leprosy, and the like.

The term “peptidomimetic” or “peptide mimetic” describes a peptide analog, such as those commonly used in the pharmaceutical industry as non-peptide drugs, with properties analogous to those of the template peptide. (Fauchere, J., Adv. Drug Res., 15: 29 (1986) and Evans et al., J. Med. Chem., 30:1229 (1987)). Generally, peptidomimetics are structurally similar to a paradigm polypeptide (i.e., a polypeptide that has a biochemical property or pharmacological activity), but have one or more peptide linkages optionally replaced by a linkage such as, —CH₂NH—, —CH₂S—, —CH₂—CH₂—, —CH═CH—(cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data, 1:3 (1983); Morley, J. S., Trends. Pharm. Sci., pp. 463-468 (1980); Hudson, D. et al., Int. J. Pept. Prot. Res., 14:177-185 (1979); Spatola et al., Life Sci., 38:1243 (1986); Harm, J. Chem. Soc. Perkin Trans 1,307-314 (1982); Almquist et al., J. Med. Chem., 23:1392 (1980); Jennings-White et al., Tetrahedron Lett., 23:2533 (1982); Szelke et al., European Appln. EP 45665 (1982) CA: 97:39405 (1982); Holladay et al., Tetrahedron Lett., 24:4401 (1983); and Hruby, Life Sci., 31:189 (1982). Advantages of peptide mimetics over natural polypeptide embodiments may include more economical production, greater chemical stability, altered specificity, reduced antigenicity, and enhanced pharmacological properties such as half-life, absorption, potency and efficacy.

Substitution of one or more amino acids within polypeptide or peptide mimetic with a D-amino acid of the same type (e.g., D-lysine in place of L-lysine) may be used to generate-polypeptides and peptide mimetics that are more stable and more resistant to endogenous proteases.

The term “receptor” refers to a molecule present on the cell membrane that has an affinity for a particular ligand. Numerous receptors are known in the art. Examples of such receptors include, but are not limited to, heparin sulfate receptor, vitronectin receptor, Vcam-1 receptor, hemaglutinin receptor, Pvr receptor, Icam-1 receptor, decay-accelerating protein (CD55) receptor, Car (coxsackievirus-adenovirus) receptor, integrin receptor, sialic acid receptor, HAVCr-1 receptor, low-density lipoprotein receptor, BGP (biliary glycoprotien) receptor, aminopeptidease N receptor, MHC class-1 receptor, laminin receptor, nicotinic acetylcholine receptor, CD56 receptor, nerve growth factor receptor, CD46 receptor, asialoglycoprotein receptor Gp-2, alpha-dystroglycan receptor, galactosylceramide receptor, Cxcr4 receptor, Glvr1 receptor, Ram-1 receptor, Cat receptor, Tva receptor, BLVRcp1 receptor, MHC class-2 receptor, and complement receptor. Fields Virology, 4^{th} edition, page 89, eds. David Knipe and Peter Howley, Lippincott Williams & Williams, Philadelphia, Pa. 2001. Flint et al., Principles of Virology, ASM Press, Washington, D.C. 2000.

The term “titer” or “tittering” refers to the concentration of antibodies in a specimen or the act of determining the number of antibodies in a specimen respectively.

II. A Method to Detect if a Candidate Agent Modulates Invasion of a Cell by an Invasin.

The invention provides a method to determine if an agent modulates invasion of a cell by an invasin. The method can be used to determine if the agent increases or decreases invasion of a cell by an invasin. The methods involve incubating an invasin that encodes a detectable label with a cell under conditions wherein the invasin can invade the cell. The detectable label can be detected within the cell to allow determination of whether or not an invasin entered the cell, and how many invasins entered a cell. The cells may be intact or lysed when the detectable label is detected. A candidate agent can be incubated in the presence of the invasin and the cell, and invasin of the cell by the invasin in the presence of the candidate agent can be determined.

The methods of the invention may be used under nearly any conditions wherein an invasin can invade a cell. For example, cells that are invaded by an invasin may be grown on plates, grown in liquid culture, grown in monolayers, or be located in vivo within the body of an organism. Large or small numbers of cells may be used within the methods of the invention. In addition, large or small numbers of invasins may be used within the methods of the invention. Methods to culture cells are well known in the art and are disclosed herein. Parameters, such as the temperature, time, growth media, pH, and atmosphere used during incubation of the cells with the invasin may be adjusted to accommodate specific cell types or invasins according to well known procedures.

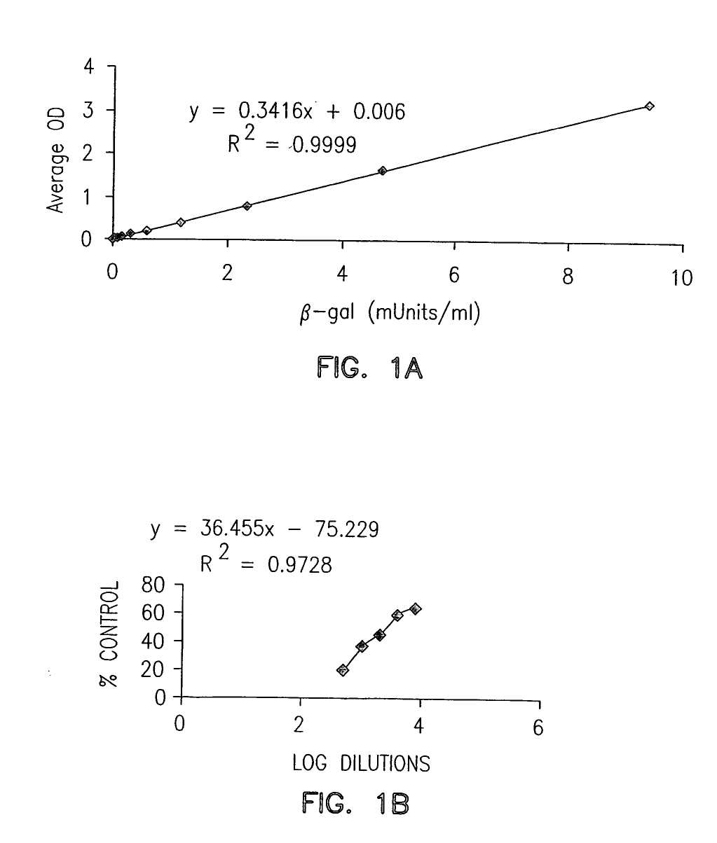

Invasion of a cell by an invasin in the presence of a candidate agent can be compared to a control experiment wherein a cell and an invasin that encodes a detectable label are incubated in the absence of a candidate agent. Alternatively, invasion of a cell by an invasin can be compared and quantitated through use of a standard curve. For example, if the detectable label is an enzyme, such as β-galactosidase, a curve may be prepared with optical density units and units of enzyme activity to determine the amount of detectable label present in a cell (see FIG. 1A). Such a calibration curve may be prepared with virtually any detectable label, such as fluorescent proteins or enzymes.

The method of the invention may be applied to a large variety of cells and invasins. Generally, the methods of the invention may be applied to any invasin that encodes a detectable label and enters a cell, such that the presence of the invasin within the cell is indicated by expression of the detectable label within the cell.

For example, the invasin may be a virus that encodes a detectable label, such as a fluorescent protein or an enzyme, which is expressed upon entry of the virus into a cell. In another example, the invasin may be a bacterium that encodes or expresses a detectable label. Those of skill in the art recognize that the methods of the invention may be applied to a broad range of invasins.

The detectable label encoded by the invasin may be encoded by an endogenous nucleic acid segment, or be encoded by an exogenous nucleic acid segment. For example, a bacterium may encode an endogenous β-galactosidase activity that can be used as a detectable label. In another example, a bacterium may be produced through recombinant nucleic acid technology that expresses an exogenous β-galactosidase activity. In other examples, many different types of viruses can be produced that encode preselected nucleic acid segments. In one example, the invention utilizes a vaccinia virus that encodes a β-galactosidase that is expressed within a cell. In another example, the invention utilizes a vaccinia virus that encodes a fluorescent protein that is expressed within a cell. A nucleic acid segment introduced into an invasin may encode one or more detectable labels. In addition, a nucleic acid segment introduced into an invasin may encode gene products other than detectable labels. For example, recombinant viruses may be constructed that express a ligand on their surface that is not expressed by the wild-type virus. Such a ligand may allow the recombinant virus to infect a cell or cells that the virus was not previously able to infect. Such ligands and recombinant viruses are known in the art. Invasins of the invention may include nucleic acid segments that are operably linked to control elements that allow constitutive expression of the nucleic acid segment, or regulated expression of the nucleic acid segment. For example, a nucleic acid segment may be operably linked to a constitutive promoter or a regulated promoter. Many examples of constitutive and regulated promoters are known in the art. Examples of regulated promoters include inducible promoters and tissue specific promoters. Thus, invasins may be constructed that express a nucleic acid segment, such a nucleic acid segment encoding a detectable label, in specific tissues or upon addition of an inducing agent that causes expression of an operably linked nucleic acid segment from an inducible promoter. Recombinant nucleic acid techniques, cloning vectors, and cellular transformation methods are well known in the art and have been described. Sambrook et al., Molecular Cloning: A Laboratory Manual, 3rd edition, Cold Spring Harbor Press, Cold Spring Harbor, N.Y. (2001).

Numerous types of cells may be utilized within the methods of the invention. Such cells can be invaded by an invasin, and allow expression of a detectable label. Naturally occurring and immortalized cells may be used within the invention. Genetically modified cells may also be used within the methods of the invention. For example, a cell may be transformed with a nucleic acid construct that directs the expression of a receptor not normally expressed by the cell. The receptor may be selected to provide for infection of the genetically modified cell by an invasin that expresses a ligand for the receptor. Accordingly, genetically modified invasins can be constructed to express selected ligands that infect genetically modified cells that express receptors for the ligands. Thus, genetically modified cells may be matched with genetically modified invasins and used within the methods of the invention. Such combinations allow one of skill in the art to produce genetically modified cells and ligands that may be used to identify candidate agents that modulate specific receptors and ligands.

Candidate agents may modulate invasion of a cell by an invasin by associating with a cell, an invasin, or combinations of cells and invasins. For example, a candidate agent may bind to a receptor on a cell that is used by an invasin to invade the cell. The candidate agent may block binding of the cellular receptor with a ligand for the receptor that is on the invasin. In another example, a candidate agent may associate with a complex formed by a cellular receptor and an invasin on a ligand to block invasion of the cell by the invasin. In another example, the candidate agent may bind to a ligand on an invasin and thereby block association of the invasin with a receptor on a cell. In yet another example, the candidate agent may bind to a complex of a ligand on an invasin and a receptor on a cell to block invasion of the cell by the invasin. Examples of candidate agents include, but are not limited to, compounds, monoclonal antibodies, polyclonal antibodies, altered antibodies, enzymes, and the like. For example, a candidate agent may be an antibody that binds to a cellular receptor, a ligand on an invasin, or complexes thereof. A candidate agent may be an enzyme that acts to modulate invasion of a cell by an invasin. For example, a protease may digest a receptor on a cell or a ligand on an invasin that is used by an invasin to invade a cell. In another example, an enzyme may glycosylate or deglycosylate a receptor or ligand used by an invasin to invade a cell. One of skill in the art can determine the receptor or receptors used by a given invasin and ascertain the type of enzyme that may serve to inactivate the receptor. Candidate agents may be peptides. Such peptides may contain naturally occurring peptide bonds, non-peptide bonds, and combinations thereof. Such peptides may be peptidomimetics. In one example, peptides may act as ligands that are bound by cellular receptors to block binding of an invasin to the receptor. The amino acid sequence of such peptides may be obtained from known amino acid sequences of receptor ligands that are used by an invasin to invade a cell. For example, the amino acid sequences of such peptides may be determined from the amino acid sequences of α-chemokine and β-chemokine that act as ligands for the Cxcr4 and Ccr5 which are used by the human immunodeficiency virus to invade a cell. A candidate agent may also be a compound. Many antiinfective compounds are known in the art. Examples of such compounds are provided by amantadine and rimantadine, which inhibit penetration and uncoating of influenza virus. Merck Manual, Merck Research Laboratories, Whitehouse Station, N.J. 17^{th} edition, eds, Beers and Berkow 1999. Merck Index, Merck Research Laboratories, Whitehouse Station, N.J., 13^{th} ed., 2001.

Many methods may be used to detect the detectable label. Chemiluminescence may be used to detect the detectable label. Briefly, the detectable label expressed within a cell invaded by an invasin can be contacted with a substrate that is acted upon by the detectable label to produce a signal that may be detected with a luminometer. For example, the following detectable labels and their substrates are provided as examples that may be used for chemiluminescent detection of cellular invasion: alkaline phosphatase with AMPPD; β-galactosidase with AMPGD; horseradish peroxidase with liminol+perborate+4-iodophenol; and xanthine oxidase with luminol+Fe EDTA (Harlow et al., Antibodies: A Laboratory Manual, page 319 (Cold Spring Harbor Pub. 1988)). Bioluminescence may be used in an analogous manner as chemiluminescence to detect a detectable label. Fluorescence may be used to detect a fluorescent protein that is expressed as a detectable label. For example, green fluorescent protein may be expressed within a cell following invasion by an invasin and detected with a fluorimeter, a fluorescent plate reader, or a fluorescent microscope. Ultraviolet or visible light may be used to detect the presence of a detectable label within a cell. Such detection methods are known in the art and are disclosed herein. The methods of the invention may be used in high-throughput

In some instances, the cells will be washed following incubation with an invasin to remove any invasin that has not invaded a cell. This washing step may reduce the amount of detectable label from invasins that have not invaded a cell and may provide a more accurate measure of cell invasion. In other instances, it is not advantageous to wash the invasin from the cells following incubation of the cells with the invasin. For example, a genetically modified virus can be used within the methods of the invention that has a cell specific promoter operably linked to nucleic acid segment that encodes the detectable marker. Such a genetically modified virus will express the detectable marker only upon invasion of a cell.

III. Methods to Determine if Invasion of a Cell by an Invasin is Decreased in the Presence of a Specimen Containing at Least One Antibody, and if an Organism was Exposed to an Invasin, such as Smallpox.

The invention provides methods to determine if a specimen contains an antibody that decreases invasion of a cell by an invasin. The methods involve incubating an invasin that encodes a detectable label, at least one cell, and a specimen suspected of containing at least one antibody under conditions wherein the invasin can invade the cell. Invasion of the cell by the invasin may be determined by detecting the detectable label within the cell. The detectable label expressed within the cells in the presence of a specimen suspected of containing an antibody is compared to a control incubation lacking the specimen, or is compared to a standard curve as described herein. If the presence of the specimen causes a decrease in the amount of detectable label detected within a cell when compared to the amount of detectable label in a cell produced in the absence of the specimen, the specimen is judged to contain an antibody that decreases invasion of the cell by the invasin. The presence of the invasion reducing antibody may be confirmed by other means known in the art, such as immunoprecipitation with a secondary antibody against the conserved region of the antibody in the specimen. Antibodies contained within a specimen may also be tittered according to the methods described herein below. In addition the methods of the invention may be used to determine the concentration of antibodies that are needed to neutralize invasion of a cell by an invasin (Burton et al., Virology, 270:1 (2000); Klasse and Sattentau, J. Gen. Virol 83:2091 (2002)). Such methods are especially useful to determine proper dosages for administration of antibody preparations. Administration of a neutralizing concentration of an antibody may avoid antibody-mediated invasion of a cell that would be promoted by administration of a subneutralizing concentration of antibodies against an invasin, such as a virus.

The methods of the invention may be used with immunogenic specimens. Examples of immunogenic specimens include, blood, serum, and tissues that may contain antibodies. Such tissues are exemplified by liver, spleen lymph nodes, and ovaries. The specimen may be obtained from an organism, such as a human, and processed to permit antibodies contained within the specimen to be used according to the methods of the invention. Processing may include homogenizing the tissues through use of a tissue homogenizer or other methods known in the art. The methods of the invention may also be used with other types of antibody containing specimens. In one example, antibody preparations may be used. Antibody preparations include pharmaceutical preparations. Generally, pharmaceutical preparations contain antibodies in a form that is able to be administered to a human or animal. Antibody preparations are often administered to humans having compromised immune systems which are often due to disease or transplant therapy. Antibody preparations are also administered in situations where it is desired to avoid using therapies that may have undesirable secondary consequences, such as during pregnancy. One example of an antibody preparation is vaccinia immunoglobulin G (VIG) as disclosed herein. VIG may be used to treat poxvirus infections, such as smallpox. Specimens containing antibodies may also be obtained from antibody secreting cells. For example, supernatant from hybridoma cultures can be collected and used within the methods of the invention to determine if hybridomas secrete an antibody that will decrease invasion of a cell by an invasin.

Accordingly, the methods of the invention may be used to screen for antibodies that decrease or neutralize invasion of a cell by an invasin. This is done by incubating a specimen suspected of containing an antibody, at least one cell, and an invasin under conditions wherein the invasin can invade the cell. Accordingly, the methods of the invention may be used to determine if an organism has been exposed to a particular invasin. The methods of the invention may be adapted to a large number of invasins through use of appropriate cells, and invasins that are constructed to encode a detectable label that is expressed upon invasion of a cell by the invasin. The methods of the invention may also be used to determine if a vaccine is able to elicit an immune response in an organism, such as a human, against an invasin. This may be done by obtaining an immunogenic specimen from the organism to which a vaccine against an invasin was administered, and incubating the specimen according to the methods of the invention. If the presence of the specimen causes a decrease in invasion of a cell by the invasin to which the vaccine was directed, as indicated by a decrease in the amount of detectable label in a cell, the vaccine may be judged to elicit an immune response.

In one example, the methods of the invention may be used to determine if a human has been exposed to smallpox. This may be done by obtaining an immunogenic specimen from a human suspected of having been exposed to smallpox and incubating the specimen with a vaccinia virus that encodes a detectable label and a cell under conditions wherein the vaccinia virus can infect the cell. A decrease in the amount of detectable label expressed within the cell in the presence of the specimen indicates that the human has been exposed by smallpox.

As described hereinabove, invasins and cells may be constructed through use of recombinant nucleic acid techniques to display preselected molecules, such as receptors and ligands, that provide for invasion of the recombinant cell by a recombinant invasin. Recombinant techniques may also be used to construct invasins that encode many types of detectable markers which are expressed upon invasion of a cell by an invasin. Thus, recombinant cells and recombinant invasins having matched receptors and ligands may be used in conjunction with the methods of the invention to determine if a specimen contains antibodies that reduce or neutralize invasion of a cell by a invasin. Recombinant cells and recombinant invasins may also be used to determine if an organism, such as a human, has been exposed to an invasin.

IV. A Method to Determine if a Vaccine Elicits an Immune Response Against Smallpox in a Human.

The invention provides methods to determine if a vaccine elicits an immune response against smallpox in a human. The methods involve incubating a vaccinia virus that encodes a detectable label, at least one cell, and an immunogenic specimen obtained from a human vaccinated against smallpox under conditions wherein the vaccinia virus can invade the cell. Invasion of the cell by the vaccinia virus may be determined by detecting the detectable label expressed within the cell. This may be done with intact cells or with cells that have been lysed. The detectable label expressed within the cells in the presence of the immunogenic specimen is compared to a control incubation lacking the specimen or to a standard curve as described herein. If the immunogenic specimen causes a decrease in the amount of detectable label detected within a cell when compared to the amount of detectable label in a cell produced in the absence of the specimen, the specimen is judged to contain an antibody that decreases invasion of the cell by the vaccinia virus.

The vaccinia virus can encode a detectable label of many different types as are known in the art and described herein. Briefly, examples of detectable labels include fluorescent proteins and enzymes that may be used in chemiluminescent methods, bioluminescent methods, visible light based detection methods, ultraviolet light based detection methods, fluorescence based methods, and the like.

The vaccinia virus may be a recombinant vaccinia virus having a cell specific promoter operably linked to a nucleic acid segment encoding the detectable label such that the detectable label is only expressed within a cell. In one example, a cell specific promoter may be operably linked to a nucleic acid segment encoding a fluorescent protein such that the fluorescent protein is expressed in the cell. Such a construct provides for detection of the detectable label using whole cells in conjunction with a fluorescence detector, such as a microscope or a fluorimeter and avoids having to lyse the cells. Accordingly, the method may be used in a a high-throughput format allowing multiple specimens to be assayed very rapidly. In another example, the vaccinia virus encodes β-galactosidase which is expressed upon invasion of the cell by the vaccinia virus. The cells may be lysed following incubation with the vaccinia virus and the specimen. The cell lysate is then contacted with a substrate for the β-galactosidase which is then converted into a product that may be detected through use of numerous means, such as a plate reader. Numerous detection means are known in the art that can be used to detect the product of substrate conversion by an enzyme that is used as a detectable label.

Many different types of cells may be used within the methods of the invention. For example, Vero cells, BSC1 cells, and HeLa cells may be used. Different cells may be used depending on the type of promoter and other control elements are operably linked to a nucleic acid segment encoding a detectable label. The combination of cells, control elements, detectable labels, and viruses can be determined without undue experimentation through use of routine cloning and screening procedures known in the art.

V. A Method to Determine if a Specimen Contains an Antibody that Binds to a Preselected Antigen.

The invention provides methods to determine if a specimen contains an antibody that binds to a preselected antigen. The methods involve incubating at least one cell, an invasin that displays a preselected antigen on its surface, and a specimen suspected of containing an antibody that binds to the preselected antigen under conditions wherein the invasin can invade the cell. Invasion of a cell by an invasin can be determined by detecting the detectable label that is expressed within a cell following invasion of the cell by the invasin. The amount of detectable label detected in a cell can be compared to a control incubation lacking the specimen suspected of containing an antibody that binds to the preselected antigen. The amount of detectable label may also be compared to a standard curve as is disclosed and described herein. A decrease in the amount of detectable label within a cell in the presence of the specimen indicates that an antibody present in the specimen decreases invasion of the cell by the invasin. An increase in the amount of detectable label in a cell indicates that the specimen contains an antibody that increases invasion of the cell.

An invasin may be produced that displays a preselected antigen on its surface through use of many methods. For example, if the invasin is a bacterium, the bacterium may be transformed with nucleic acid segment that encodes the preselected antigen such that the preselected antigen is displayed on the surface of the bacterium. The preselected antigen may naturally be displayed on the surface of the bacterium or the preselected antigen may be operably linked to a signal sequence that causes the preselected antigen to be directed to the surface of the bacterium. In another example, an enveloped virus having a preselected antigen on its surface may be produced by constructing a recombinant virus that encodes the preselected antigen (U.S. Pat. Nos. 5,723,287 and 6,297,004; Ohishe et al., J. Gen. Virol., 8111439 (2000)). Upon invasion of a packaging cell by the virus, the preselected antigen may be expressed an deposited on the surface of the packaging cell. Upon viral budding of the enveloped virus from the packaging cell, the enveloped virus will become coated with the preselected antigen. The preselected antigen may naturally be displayed on the surface of the packaging cell. The preselected antigen may also be operably linked to a signal sequence that causes the preselected antigen to be displayed on the surface of the packaging cell. In another example, a recombinant packaging cell may be constructed that expresses the preselected antigen on its surface. The preselected antigen may be operably linked to a signal sequence that causes the preselected antigen to be displayed on the cell surface.

Many preselected antigens may be used within the methods of the invention. For example, preselected antigens that correspond to peptides used as subunit vaccines may be used. In another example, preselected antigens may be used that correspond to peptides expressed by viruses that act as ligands for cellular receptors and facilitate invasion of the cell by the virus. Such ligands are known in the art and examples of such ligands are disclosed herein. It has been suggested that these ligands serve as a main target for a humoral response against an invasin (Burton et al., Curr. Top. Microbiol. Immunol., 260:109 (2001)).

Recombinant methods to construct cells and viruses that encode and express preselected antigens are known in the art. Sambrook et al., Molecular Cloning: A Laboratory Manual, 3rd edition, Cold Spring Harbor Press, Cold Spring Harbor, N.Y. (2001). In addition, methods to transform cells and selected for cells that have been transformed are known in the art. Briefly, a nucleic acid segment encoding a preselected antigen may be chemically synthesized or isolated from nature. This nucleic acid segment may then be cloned into a cloning vector or an expression vector through use of restriction endonucleases and ligase. Numerous vectors and materials for cloning are known in the art and are commercially available. (Clontech, Palo Alto, Calif.; Stratagene, La Jolla, Calif., New England Biolabs, Beverly, Mass.). The recombinant DNA molecule may be introduced into a cell through calcium phosphate precipitation, ballistic transformation, liposome transformation, and the like. Transformed cells may be selected for through use of selection markers, such as streptomycin, tetracycline, ampicillin, and the like. Analogous methods may be used to construct viruses and other types of invasins.

VI. A Method to Determine if Invasion of a Cell by an Invasin is Decreased in the Presence of a Monoclonal Antibody that Binds to a Receptor Used by the Invasin to Invade the Cell.

The invention provides methods to determine if invasion of a cell by an invasin is decreased in the presence of a monoclonal antibody raised against a cellular membrane that binds to a receptor used by the invasin to invade a cell. The methods involve immunizing an animal with a cellular membrane prepared from a cell that is invaded by an invasin. The immunized animal is then processed to produce hybridomas that secrete monoclonal antibodies. Many of these monoclonal antibodies will recognize components of the cellular membrane. Methods to produce monoclonal antibodies are well known in the art and have been described. Harlow et al., Antibodies: A Laboratory Manual, page 319 (Cold Spring Harbor Pub. 1988). The plethora of monoclonal antibodies produced by the hybridomas may then be screened by incubating at least one cell, an invasin that encodes a detectable label, and a monoclonal antibody under conditions wherein the invasin can invade the cell. Invasion of a cell by the invasin may be determined by detecting the detectable label within the cell. Detection of the detectable label may be done according to methods known in the art and described herein. A monoclonal antibody that blocks invasion of a cell by an invasin may be identified based on the ability of the antibody to cause a decrease in detectable label in a cell when compared to a control incubation lacking the antibody, or a control curve as described herein.

Binding of the antibody to a specific receptor used by an invasin to invade a cell may be additionally confirmed by isolating the specific receptor and then showing that the receptor participates in invasion of a cell by an invasin. Many methods may be used to demonstrate the participation of a receptor in cell invasion by an invasin. For example, the gene encoding the receptor may be transferred to a cell that previously did not express the receptor. The transformed cell may then be shown to be invaded by an invasin that could not invade the same cell type lacking the receptor. Alternatively, the gene encoding the receptor used by the invasin to invade the cell may be mutated or knocked-out to produce a non-functional receptor or eliminate expression of the receptor. Cells expressing a non-functional receptor, or cells that do not express the receptor may then be shown resistant to invasion by the invasin.

Methods to knock-out genes are commonly used and are known in the art. Briefly, one method to knock-out a gene that expresses a nucleic acid sequence encoding a receptor is to digest the nucleic acid segment encoding the receptor with restriction enzymes such that a portion of the nucleic acid sequence encoding the receptor is deleted. The deleted portion is typically located in the middle to the nucleic acid sequence that encodes the receptor. A selection marker may be ligated into the deleted portion of the nucleic acid segment that encodes the receptor to produce a recombinant molecule. The selection marker often encodes drug resistance. The recombinant molecule having a selection marker flanked by nucleic acid sequences that code for portions of a receptor may be introduced into a cell through use of numerous art recognized methods. The recombinant molecule will replace the wild-type nucleic acid sequence encoding the receptor in the cell by homologous recombination and will thereby knock-out expression of the receptor. Cells having a knocked out receptor may be selected through use of the selection marker carried in the recombinant molecule. The knock-out may be confirmed by nucleic acid hybridization techniques known in the art, such as Southern blotting or nucleic acid sequencing.

The receptor used by the invasin to invade the cell can then be identified through use of the identified antibody. Numerous techniques have been used to identify viral receptors and coreceptors, including use of monoclonal antibodies raised against cell surface proteins, gene and/or gene transfer strategies, the virus overlap blot assay, and screening the functions of cell surface proteins that are highly related to known viral receptors. Such viral receptors and coreceptors have been reported. Fields Virology 4^{th} edition, page 89, eds. David Knipe and Peter Howley, Lippincott Williams & Williams, Philadelphia, Pa. 2001.

Monoclonal antibodies that bind to specific cell surface proteins and block viral infection have been used extensively to identify viral receptors. Receptors identified by this approach include CD4 for HIV-1 (Dalgleish et al., Nature, 312:763 (1984); Kaltzmann et al., Nature, 312:767 (1984); Maddon et al., Cell 47:333 (1986)), intercellular adhesion molecule (ICAM-1) for subgroups of rhinoviruses (Greve et al., Cell, 56:839 (1989); Staunton et al., Cell, 56:849 (1989)) MHVR/Bgp1(a) for mouse hepatitis virus A59 (Dveksler et al., J. Virol., 65:6881 (1991)) CAR for cozsackie B viruses, and adenoviruses (Bergelson et al., Science, 275:1320 (1997); Tomko et al., Proc. Natl. Acad. Sci., 94:3352 (1997)) CD46 for measles virus (Dorig et al., Cell, 75:295 (1993)), and CR2 for Epstein-Barr virus (Fingeroth et al., Proc. Natl. Acad. Sci., 81:4510 (1984); Frade et al., Proc. Natl. Acad. Sci., 82:1490 (1985); Nemerow et al., J. Virol, 55:347 (1985)). Numerous other receptors and coreceptors have been identified through use of monoclonal antibodies.

Gene-transfer and cDNA-transfer methods have been used to clone viral receptors and coreceptor genes. These approaches involve preparing genomic DNA libraries or cDNA libraries from donor cells that express a viral receptor and then introducing these molecules into a recipient cell type that lacks the receptor. Transduced cells that express the receptor are then be identified. Viral receptors and coreceptors that were identified by gene transfer or cDNA transfer include PVR for polioviruses (Mendelsohn et al., Cell, 56:855 (1989)) PiT-1 for gibbon ape leukemia viruses/subgroup B feline leukemia viruses/simian sarcoma-associated viruses (O'Hara et al., Cell. Growth Diff., 1:119 (1990)), ATRC1/MCAT-1 for ecotropic MLV (Albritton et al., Cell 57:659 (1989)). Numerous other viral receptors were identified through use of gene-transfer and cDNA-transfer methods.

Several viral receptors, including low-density lipoprotein receptor (LDLR)-related receptor proteins for minor subgroups of rhinoviruse (Hofer et al., Proc. Natl. Acad. Sci., 91:1839 (1994)) and alpha-dystroglycan for lymphocytic choriomeningitis virus and lassa fever virus (Cao et al., Science, 282:2079 (1998)), have been identified using the virus overlay protein blot assay. This assay utilizes virus binding to receptors that have been immobilized on a membrane following SDS-polyacrylamide gel electrophoresis.

Accordingly, numerous methods may be used to identify a receptor used by an invasin to invade a cell. In addition, the methods of the invention may be used with many different invasins that are known in the art and disclosed herein.

VI. Methods to Determine if an Agent Modulates Antibody-Mediated Infection.

The invention provides methods to determine if an agent modulates antibody-mediated invasion of a cell by an invasin. The methods involve incubating at least one cell, an invasin that encodes a detectable label and that utilizes antibody-mediated invasion to invade the cell, a subneutralizing concentration of antibodies that recognize the invasin, and a candidate agent. Invasion of the cell by the invasin may be determined by detecting the amount of detectable label in a cell. The ability of the candidate to modulate antibody-mediated invasion of a cell may be determined by comparing invasion of a cell in the presence and in the absence of the candidate agent. This comparison may be done through use of control experiments lacking the candidate agent or through use of a standard curve, as described herein.

The effect of the candidate agent on antibody-mediated invasion may be determined through use of control experiments that include no antibody, a subneutralizing concentration of antibody, and a neutralizing concentration of antibody. If the candidate agent does not affect antibody-mediated invasion, invasion of a cell by an invasin will be the same in the presence of the candidate agent when no antibodies are present. However, if the candidate agent does affect antibody-mediated invasion of a cell, invasion of a cell in the presence of a antibodies will be increased or decreased in the presence of the candidate agent.

Antibody-mediated enhancement of infection is thought to occur at subneutralizing concentrations of antibodies (Halstead, Progr. Allergy, 31:301 (1982); Hawkes and Lafferty, Virology, 33:250 (1967); Morens et al., Microb. Pathog., 3:231 (1987); Parren and Burton, Adv. Immunol. 77:195 (2001)). Enhancement is very sensitive to the target cell and is dependent on the types and expression levels of Fc and/or complement receptors on the target cell surface. A classical example is enhancement of dengue virus infection, which is dependent on the interaction between virion-bound antibody and Fc receptors expressed on the target cell. In typical assays, neutralization is observed at relatively high concentrations, whereas enhancement is observed at lower concentrations. Using neutralizing antibodies against dengue virus, it was shown that the monoclonal antibody concentration maximizing enhancement was predicted by the neutralization titer (Morens et al., Microb. Pathog., 3:231 (1987). The balance between enhancement and neutralization was also shown to be dependent on the incubation time of virus and antibody. Hawkes and Lafferty, Virology, 33:250 (1967) A shift of the dose response curve as time progressed was observed, and enhancement as well as neutralization occurred at increasingly lower concentrations. Presumably, at early time points, attachment of a small number of antibodies leads to enhancement, and as the reaction progresses, more antibodies attach, eventually leading to neutralization of the virion. This indicates that enhancement and neutralization are two different biological outcomes of the interaction of an antibody with virus at different levels of occupancy.

For HIV-1, both Fc receptor-mediated and complement-mediated antibody-dependent enhancement of infection have been described (Homsy et al., Science, 244:1357 (1989); Lund et al., J. Virol., 69:2393 (1995); Mascola et al., AIDS Res. Hum. Retroviruses, 9:1175 (1993); Robinson et al., Lancet, i:790 (1988); Schutten et al., Scand. J. Immunol., 41:18 (1995); Takeda et al., Science, 242:580 (1988)). In Fc receptor-mediated enhancement, Fc receptor-mediated endocytosis of virion-antibody complexes may lead to the internalization of virus and infection. An alternative is that binding to Fc receptors stabilizes the interaction of virion and target-cell, permitting interaction of the envelope spike and virus receptor at low antibody coating of the virion (Connor et al., Proc. Natl. Acad. Sci., 88:9593 (1991)). At higher coating, this interaction may be inhibited. Complement receptor 2 (CD21) may mediate enhancement of HIV-1 infection via several proposed mechanisms, either by acting as a receptor for HIV-1 opsonized with antibody and complement or by increasing virus binding to the cell due to an interaction of CD21 with opsonized virus (Boyer et al., Scand. J. Immunol., 36:879 (1992); Lund et al., J. Virol., 69:2393 (1995)). In addition, virion-bound C1q may directly interact with receptors on the host cell, leading to enhancement of infection (Prohaszka et al., AIDS, 11:949 (1997)). In some cases, such as HIV-1, the enhancement does not require the Fc part of the antibody molecule (Sullivan et al., J. Virol., 72:6332 (1998b)). The low-level coating may trigger conformational changes in the envelope that, for example, favor fusion between the virion and target cell, or such coating may nonspecifically reduce repulsion between the virion and target cell surface. A similar mechanism of antibody-mediated enhancement of infection has been described for Sindbis virus (Flynn et al., Virology, 166:82 (1988)).

The phenomenon of enhancement indicates that antibodies bind to virions at subneutralizing concentrations and is therefore a strong argument for the importance of antibody occupancy in virus neutralization and neutralization following multi-hit kinetics. High levels of occupancy lead to neutralization, whereas, in the presence of permissive target cells, low levels of occupancy may lead to enhancement of infection.

Accordingly, the methods of the invention may be used to identify an agent that modulates antibody-mediated invasion of a cell by an invasin.

VIII. A Method to Determine if an Antibody Mediates Transport of an Invasin Across a Cell Monolayer.

The invention provides methods to determine if an antibody mediates transport of an invasin across a cell monolayer. The method involves adding an invasin that encodes a detectable label to a first side of a cell monolayer, adding an antibody to a second side of a cell monolayer, and determining if the antibody mediates transport of the invasin across the cell monolayer. Transport of the invasin across the cell monolayer may be determined by detecting the amount of detectable label in a cell of the monolayer and on the second side of the monolayer. The ability of the antibody to modulate transport across the monolayer may be determined by comparing transport of the invasin in the presence and absence of an antibody. This comparison may be done through use of control experiments lacking an antibody or through use of a standard curve, as described herein. The invention further provides a method to determine if a candidate agent can modulate antibody mediated transport of an invasin across a cell monolayer. The method involves conducting the method as described above with the additional step of adding a candidate agent to the first side or the second side of the cell monolayer, and determining if the candidate agent increases or decreases antibody mediated transport of the invasin across the monolayer. The effect of the candidate agent on antibody mediated transport may be determined by comparing antibody mediated transport of an invasin across a cell monolayer in the presence and absence of the candidate agent.

In mucosal transmission, antibodies are thought to bind intracellularly to transcytosing viruses, leading to neutralization or redirection of the transcytosing virus. Polymeric IgA and IgM are actively transported over the mucosal epithelium after binding to the polymeric Ig receptor and are thought to contact and neutralize transcytosing viruses during transport. Sendai virus added to the apical side of an epithelial cell monolayer was shown to interact intracellularly with polymeric IgA added basolaterally, and viral titers were reducted in cellular supernatant and lysate (Manzanec et al., Virus Res., 23:1 (1992); Fujioka et al., J. Exp. Med., 188:1223 (1988)). It has been demonstrated that influenza virus an be neutralized through a similar mechanism (Manzanec et al., Virus Res., 3:1 (1995)). It has also been shown that transcytosis of primary HIV-1 isolates over tight epithelium can be blocked by dimeric immune IgA and IgM (Bomsel et al., Immunity, 9:277 (1998)). The basolaterally internalized antibodies met transcytosing virions in an intracellular compartment, thereby redirecting the virions back to the mucosal compartment (Bomsel et al., Immunity, 9:277 (1998)). Thus, cell monolayers and assay conditions that provide for transport of an invasin across a cell monolayer may be prepared according to the guidance provided by the art and described herein.

Any invasin, candidate agent, detectable label, cell, or detection method described herein may be utilized within the methods of the invention. In addition, those of skill in the art realize that many cell types may be used to prepare a cell monolayer. Cells that form tight junctions may be especially useful for the preparation of cell monolayers. Such cells include intestinal epithelial cells.

IX. A Method to Assay Invasin Load In Vivo.

The invention provides methods to determine invasin load in vivo. The methods involve infecting an organism with an invasin that encodes a detectable label to produce an infected organism, and then detecting the detectable label in specimens obtained from the infected organism. The detectable label indicates the presence of the invasin in the specimen obtained from the infected organism. Thus, the methods of the invention allow the spread of an invasin to be followed over time, as well as the concentration of invasin. Such methods are useful for following the progression of a disease caused by an invasin and for determining appropriate treatment schemes and dosages for therapeutics that can be administered to an organism to combat an invasin. Examples of specimens that may be obtained from the organism include, liver, spleen, kidney, lung, intestine, blood, lymph nodes, ovaries, PBL, and the like. Also, numerous organisms may be used within the methods, such as avians, cats, dogs, sheep, horses, ferrets, mink, beavers, muskrats, cattle, pigs, rabbits, monkeys, rats, mice, and the like.

The organisms may have a functional immune system or have a non-functional immune system. Examples of organisms with non-functional immune systems include SCHD and nude mice. Organisms may genetically lack a functional immune system, or the immune system of a normal organism may be destroyed or disabled prior to use of the organism in the methods of the invention. Methods to destroy or disable the immune system of an organism include, but are not limited to, use of radiation, chemicals, or combinations thereof.

Numerous types of invasins may be used within the invention. Examples include, viruses, bacteria, nematodes, and the like. Such invasins are known in the art and are described herein. Detectable labels and detection methods may be selected from those described herein, or others known in the art that are adapted to the methods of the invention. Invasins may be constructed through use of common recombinant nucleic acid techniques to encode a detectable label, and provide expression of the detectable label when the invasin invades a cell.

The organism may be infected with an invasin following administration of a therapeutic, such as a vaccine, an antibody preparation, a monoclonal antibody, a polyclonal antibody, an altered antibody, an enzyme, a peptide, a compound, a pharmaceutical composition, or any combination thereof to the organism. Accordingly, the methods of the invention may be used to determine if administration of a therapeutic prior to infection with an invasin can modulate invasin dissemination and concentration throughout the organism. A therapeutic such as those listed above may be applied to an organism after the organism is infected with an invasin. Thus, the methods of the invention can be used to determine if administration of a therapeutic to an organism after the organism is infected with an invasin can modulate invasin dissemination or concentration within an organism.

X. Kits.

The invention provides kits containing packaging material, a vaccinia virus that encodes β-galactosidase, and at least one cell that can be invaded by the vaccinia virus. The invention provides kits containing packaging material, a vaccinia virus that encodes a fluorescent protein, and at least one cell that can be invaded by the vaccinia virus. A cell contained in a kit of the invention may be a cell that can be infected by a vaccinia virus and expresses a fluorescent protein, β-galactosidase, or both β-galactosidase and a fluorescent protein. A particular cell that may be contained in a kit of the invention is a HeLa cell. HeLa cells and other types of cells can be obtained from the American Type Culture Collection, Manasses, Va. Numerous fluorescent proteins may be encoded by a vaccinia virus contained in a kit of the invention. Such fluorescent proteins are known in the art and have been disclosed herein.

### EXAMPLE I

I. Vaccinia Immune Globulin Preparations

Two vaccinia immune globulin preparations were tested. They were both prepared from the same pooled plasma obtained from military personnel vaccinated and boosted with the Dryvax vaccine. The first preparation, Baxter VIG (16.5%), was manufactured in 1994 by cold-alcohol precipitation based upon the Cohn-Oncley method for purification of immune globulins. The second preparation, VIGIV (5%), was manufactured using similar methods, but by a different manufacturer (Massachusetts Biological Laboratories), in 2001.

II. Plaque Reduction Neutralization Titer

Two PRNT assays were compared to the new reporter gene method. One assay was performed by BioReliance Corporation (Rockville, Md.), and sponsored by Dynport Vaccine Company. VERO 76 cells (ATCC CRL-157), at passage 38-60, were seeded onto 6-well plates in EMEM, with 10% FBS, and 1% (v/v) L-glutamine. The seeding density was 5×10⁴/ml 72 hours prior to inoculation. VIG and VIGIV specimens were heat-inactivated at 56° C. for 30 minutes. Vaccinia virus was obtained from a working bank, derived from a New York City Board of Health strain, and was added to serially diluted test specimens at a concentration of 500 PFU/ml. The tubes containing virus mixed with test specimens were incubated on a shaker set at 120 rpm in a humidified CO₂ incubator at 360 for 90 minutes. Each assay included titration of viral stock, positive control sera, and negative control (media only; no virus) wells. Media was carefully removed from VERO 76 cells, and the 200 μl of the neutralized virus/specimen mixtures, or controls, were added to the plates, in triplicate for each dilution. The plates were placed on a plate rocker in a humidified CO₂ incubator at 36° C. for 60 minutes (adsorption period). After adsorption, a 2 ml agarose overlay consisting of 35% Seakem ME Agarose (1.8% solution), and 65% (v/v) overlay media (2×EMEM, 2% L-glutamine, 1% NEAA, 10% FBS, and 0.1% neomycin) was added to each well. After 48 hours incubation in a humidified CO₂ incubator, 2 ml of a second agarose overlay was added, consisting of 62% overlay medium (see above), 35% Seakem ME Agarose (1.8% solution), and 0.333% Neutral Red Solution (Biologos, Inc., Montgomery Ala.). After 24 hours of further incubation as before, plates were removed and protected from light before counting. Plaques were counted using a white light transilluminator. The 50% neutralization titer for each specimen was calculated using the Minitab Probit statistical analysis, and the antibody titer value was reported.

The second PRNT assay was conducted by Acambis (Cambridge, Mass.). In that assay Vero cells (P/N CB0006) were seeded in 12-well culture plates at 3×10⁵ cells/ml in EMEM with 5% FBS (heat-inactivated, Hyclone, Logan Utah) 24 hr prior to their use in the plaque assay. The vaccinia virus used in the assay was derived from Dryvax vaccine (New York City Board of Health strain) by two passages in MRC-5 cells. The virus was diluted to 1,300±100 pfu/ml in MEM (with 10% heat-inactivated FBS, 20 mM HEPES buffer, 1,000 units/ml penicillin and 1 mg/ml streptomycin). Test articles and controls are serially diluted (two-fold dilutions) to 2× final concentrations and mixed at a 1:1 ratio with the virus. Rabbit anti-vaccinia (WR) serum serves as a positive neutralization control, whereas dilution medium was used as a negative control. Virus/antibody mixtures are incubated in a 4° C. refrigerator overnight (18±2 hr). 100 μl of virus/antibody mixtures were added to seeded Vero cells in duplicates, and the plates were incubated for 1 hr at 37° C. in a CO₂ incubator with intermittent rocking, followed by addition of 1 ml overlay medium (MEM with 10% FBS plus antibiotics). Plates were incubated at 37° C., in a CO₂ incubator for 2 days (48±4 hr). Medium was removed from all wells and replaced with 1 ml of 1% crystal violet in 70% methanol for ≧30 min to fix and stain the cells and to inactivate virus. Plates were washed in tap water and air-dried. Plaques were scored using a stereomicroscope over a light box. The neutralization titer was defined as the highest dilution of test article that reduces the average pfu/well by ≧50% from average pfu number in all negative control wells run in the assay.

III. β-Galactosidase-Based Vaccinia Neutralization Assay (β-Gal Assay)

This assay was conducted in HeLa cells (CCL-2) obtained from ATCC (Manassas, Va.). Cells are maintained in EMEM (Mediatech, Herdon, Va.) supplemented with 10% FCS (heat-inactivated, Sigma-Aldrich, St. Louise, Mo.), 2 mM L-glutamine, and Earle's BSS adjusted to contain 1.5 g/L sodium bicarbonate, 0.1 mM non-essential amino acids, and 1.0 mM sodium pyruvate. HeLa cells were used at ≦14 passages. Preliminary experiments demonstrated that in this assay HeLa cells performed better than BSC1 or Vero cells (higher expression of β-Gal per cell).

Two recombinant viruses were tested in this assay. vSC8 expresses β-Gal under the control of a late vaccinia promoter P11, and vSC56 expresses β-Gal under the control of a synthetic E/L promoter (Chakrabarti et al., Biotechniques, 23:1094 (1997)). Virus titrations were conducted in BSC1 after infection of cells for 2 days with serial dilutions of the viral stock. The plaques were counted after addition of 0.1% crystal violet solution in 20% ethanol. At least two titrations were conducted for each viral stock. To disperse aggregates, viral stock was either sonicated (15-30 seconds on ice) or treated for 30 min at 37° C. with trypsin (0.25% in HBSS) in calcium/magnesium-free PBS at 1:1 volume ratio. Each neutralization assay included virus titration (3-4 serial dilutions spanning the intended MOI)

In order to confirm that the measured fl-Gal activity was due to newly expressed proteins following viral infection, HeLa cells were treated with cytosine arabinose (Ara C) (Sigma) at a concentration of 40 μg/ml after virus infection. Ara C blocks transcription dependent on the vaccinia late promoters (Chakrabarti et al., Biotechniques, 23:1094 (1997)).

Virus was incubated with test specimens or with positive and negative controls serially diluted (5×2-fold dilutions within the anticipated dynamic range) in EMEM/10% FCS (heat-inactivated). 150 μl each of virus (at MOI of ≦0.05/ml) and antibodies were mixed in microtubes (Sarstedt) and incubated at room temperature for 1 hr with gentle mixing. The virus/antibody mixtures (300 μl) were then added to 1.5×10⁶ HeLa cells/tube and mixed gently. The cell/antibody mixtures were incubated for 2 hr at 37° C. in a CO₂ incubator. Media was added to adjust the cell concentration to 1×10⁶ cells/ml, and the cell/virus mixtures were distributed to wells in 96-well flat bottom plates at a concentration of 2×10⁵ cells/well (4-6 replicates per antibody dilution). The set up has also been simplified by preincubating the virus with antibodies at 37° C. for 1 hr and dispensing the virus/antibody mixtures into 96-round bottom plates containing 2×10⁵ HeLa cells per well (4-6 replicates per antibody dilution). FDA interim Standard (VIGIV lot#1 from MBL/Dynport) was routinely used as a positive neutralization control, while pooled sera from vaccinia naïve individuals, pre-immune animal sera, and serial dilutions of BSA solution (5%) were used as negative controls as needed. Virus titration plates and virus neutralization plates are incubated for an additional 16 hr at 37° C. in a CO₂ incubator. Twenty μl of 20% solution of the detergent IGEPAL CA630 (Sigma-Aldrich) was added to each well with vigorous mixing to lyse the cells. At this point, the plates may be frozen at −70° C. for later use.

Each β-Gal enzymatic assay included generation of a standard curve with a recombinant β-galactosidase enzyme protein (Roche Diagnostics, 300 U/mg). Enzyme stock (5 mg) was resuspended in 0.5 ml PBS+0.5 ml glycerol for a concentration of 1,500 units/ml. Aliquots were stored at −20° C. A β-Gal working solution (15 U/ml) was prepared in 50% glycerol solution (in PBS) containing BSA (400 μg/ml). Aliquots were stored at −20° C. The enzymatic reaction was conducted in Z buffer (98.55 ml 0.1M sodium phosphate, 1 ml 1M KCL, 0.1 ml 1M MgSO₄, 0.35 ml β-mercaptoethanol (Sigma)). CPRG substrate stock (Roche Diagnostics) was prepared at 4 mg/ml in 0.1M sodium phosphate (pH 7.0).

Plates were thawed completely and were kept at 4° C. for the duration of the assay set-up. The enzymatic reactions were conducted in 96-well Immulon 2 plates (Dynatech). Blank wells (to blank the machine) contain 80 μl Z buffer and 20 μl of PBS. A β-Gal standard curve was prepared according to the following procedure. A β-Gal working solution (15 units/ml) was diluted 1:100 (to 0.15 U/ml=150 mU/ml), followed by a serial two-fold dilutions in tubes containing Z buffer and PBS (at a 4:1 ratio). In each assay plate, the first two columns (1+2) were used for the β-Gal standard curve at concentrations ranging from 18.75 to 0.3 mU/ml (in duplicates). The last 2 wells (1H, 2H) were used for Blank wells. For the experimental groups, all the wells in the odd-numbered columns contain 180 μl of PBS and all the even-numbered columns contained 90 μl of Z buffer.

From each well of infected cells (after thawing of plates they were kept at 4° C. for the duration of the assay), 20 μl were transferred to a well (in the Immulon 2 plate) containing 180 μl PBS (1:10 dilution), mixed by multiple pipetting, and 10 μl was transferred to a well containing 90 μl Z buffer (final dilution 1:100).

Twenty μL of the CPRG substrate (freshly made) was added to all wells in the standard curve columns and the even-numbered columns (containing diluted specimens in Z buffer), and the plates were incubated in the dark at room temperature for 30 min. 50 μl of 1M Na₂CO₃ was added to stop the reaction, and the plates were read by an ELISA reader at 575 nm wavelength. The machine was blanked on the “Blank wells” (see above) and the data was saved.

Microsoft Excel was used to generate a standard curve for each plate. The equation (y=bx+a) of the linear portion of the standard curve and the initial dilution factor (1:100) were used to convert the optical density (OD) values of each specimen to β-Gal activity in mU/ml, and the mean±SD for each group of replicates were calculated.

Using the β-Gal activity in the virus only plate (at 0.05 pfu/cell) as 100% (un-inhibited control), each experimental group was expressed as % of control. Microsoft Excel was used to plot the % of control values for the serial dilutions of a given antibody preparation vs. log dilutions. The equation of the curve was used to calculate the 50% inhibitory dilution (ID₅₀). In the case of VIG and VIGIV, the starting concentrations were known so the 50% inhibitory dilutions could be converted to ID₅₀ in μg/ml.

IV. Generation of QC Charts and Statistical Analysis

Data were analyzed and control charts were generated using JMP statistical analysis software. Distributions were tested for normality prior to analysis and charting. Results of repetitive tests of specimens MPH (5%) and Baxter (16.5%) are shown in the Shewhart charts (QC charts). The limits indicated on the charts are the 2-kappa limits calculated from the data, representing the approximate 95% upper and lower confidence intervals. The Baxter specimen appeared to have much greater variability than the MPH specimen. ANOVA statistical analysis (for non-paired values) determined that the difference in ID₅₀ values for VIG and VIGIV preparations were highly significant to (p<0.0001)

V. Feasibility to Validate and Transfer the Assay

Assay performance was evaluated and included repeatability and variability of the test under the current standard operating procedure. Good agreement within and between the six replicate wells for a given specimen tested in a single plate was observed. Provided that appropriate outlier rejection criteria are incorporated in the test, within-specimen variability is unlikely to be a major contributor to the variability of the assay. The β-Gal standard curves showed good agreement among multiple plates. Furthermore, the variability of the standard curve between tests was small, so that appropriate limits on performance can be established for specific conditions.

The results to date indicate the feasibility of validating and transfering the assay to other laboratories. Criteria for outlier rejection and specifications for quality control of the test are being evaluated. Appropriate limits to be placed on the performance of the virus control and standard curve for β-Gal activity, the variability of replicates at each specimen dilution within a given test, and the linearity of the dilution curve for the reference standard are under considerations.

VI. EGFP-Vaccinia (vNB009) Neutralization Assay

HeLa cells are the same as those used in the $-Gal assay and were maintained in the same media. Assays were performed with cells ≦14 passages. The virus used in this assay was vNB009 (Bleckwenn, Biotechnology Progress, (2002)). The virus contains the gene for enhanced green fluorescent protein (EGFP) under the control of a T7 promoter as part of the VOTE expression system (Ward et al., Proc. Natl. Acad. Sci., 92:6773 (1995)). Briefly, addition of the inducing agent, IPTG, allowed production of T7 RNA polymerase, which binds to the T7 promoter controlling EGFP expression, thereby expressing the recombinant protein.

The viral stock for vNB009 was titered in BSC-1 cells after infection of the cells for 3 days with serial dilutions of the stock. Duplicate wells were infected for each serial dilution and the assay was performed in triplicate. The titration assay utilized incubation of the viral stock with 1× trypsin (made fresh from 10× stock, 2.5 mg/ml) prior to infection to break up clumps of virus. Plaques were counted after 0.15% crystal violet 11% formaldehyde 0.05% ethanol solution staining.

The neutralization assay is initiated by serially diluting the FDA standard and test articles in media (DMEM without serum or phenol red (Biofluids, P152-500) supplemented with 4 mM L-glutamine 1 mM IPTG). The absence of phenol red in the media avoids interference with the fluorescence detection of the EGFP protein. Dilutions in the range of 1:500 to 1:16000 for VIG products were used to achieve proper neutralization and expression levels.

Virus vNB009 was prepared by incubating an appropriate aliquot of the stock with an equal volume of 1× trypsin, made fresh from 10× trypsin, at 37° C. for 30 minutes with vigorous vortexing every 10 minutes. At the end of the incubation, virus was diluted serially with media to obtain final multiplicities of infection (MOI) between 0.1 and 0.0125 pfu/cell (control virus titration). Equivalent volumes of diluted virus at MOI 0.05 were mixed with each dilution of the neutralization specimens. All specimens were incubated at room temperature for 1 hour with periodic shaking.

HeLa cells were prepared by harvesting from confluent T162 cm² tissue culture flasks using cell dissociation solution (Hanks' Based, Invitrogen). Viable cells were counted and distributed to polypropylene tubes with 1.5×10⁶ cells per tube. They were then centrifuged at 1000 g for 5 minutes and supernatant was aspirated. Cells were then resuspended in 300 μl of each neutralization specimen or viral titer specimen. These tubes were incubated at 37° C. for 2 hours with periodic shaking. At the end of the incubation, 1.2 ml DMEM+4 mM L-glutamine+1 mM IPTG+10% FBS without phenol red was added to each tube and mixed. Aliquots of 200 μl of each specimen were dispensed into six wells (outside wells left empty) of a black walled, tissue culture treated 96-well plate (Corning Costar 3603). The plates were incubated in 37° C. humidified incubator with 95% air, 5% CO₂.

At 44 hours post infection (hpi), the plates were removed from the incubator. Standards of recombinant EGFP were prepared in DMEM+4 mM L-glutamine+1 mM EPTG+10% FBS without phenol red. 200 μl of each standard (0-4 μg/ml rEGFP) was placed in extra wells of one plate. Each plate was read using a SpectraMax Gemini XS fluorescent plate-reader (Molecular Devices). Well-scan mode with 9 spacial readings per well and 6 reads per well were performed at exitation 485 nm and emission 512 nm with auto cut-off filter at 495 nm. The instrument reports the average reading per well and the average and standard error of all six wells for each specimen are used for data analysis. Statistical analysis was performed in the same manner as was used with the β-Gal assay where percent of control versus log dilution ratio was used to obtain ID₅₀ values.

### EXAMPLE II

I. Selection of Recombinant Vaccinia Virus, Cell Substrate, and Establishment of Neutralization Assay.

When selecting a recombinant vaccinia expressing a reporter gene for a single-round of infection, it was reasoned that an enzyme-encoding gene would provide an augmented sensitivity compared with other reporter genes, since each enzyme molecule could convert several substrate molecules within the time frame of the assay. A panel of β-galactosidase expressing recombinant vaccinia viruses (all in the WR background) under the control of either vaccinia promoters or a synthetic E/L promoter was constructed in the laboratory of Bernard Moss (NIAID, NIH) (Chakrabarti et al., Biotechniques, 23:1094 (1997)). Two of these recombinant viruses were selected, vSC8 and vSC56, to study. Virus titrations were conducted in HeLa cells using untreated or Ara C treated cells. β-Gal activity was measured in the cell extracts after 16-18 hr infection as described above.

In each enzymatic assay, a β-Gal standard curve was generated with recombinant enzyme protein, and the equation fitting the standard curve was used to convert absorbance (OD at 575 nm) to β-Gal activities in mU/ml (FIG. 1 and Table 1). To prepare the standard curve, serial dilutions of a galactosidase working solution (from 18.75 to 0.15 mU/mil) were added to a 96-well plate in duplicates. CPRG substrate was added for 30 min at room temperature in the dark and the reaction was stopped by 1M Na₂CO₃ solution. The plate was read by an ELISA reader at 575 nm wavelength and an Excel program was used to generate the linear curve. The standard curve had an r² value of ≧0.999.

In vSC8 the β-galactosidase gene is under the control of a late vaccinia promoter (P11). Therefore, expression was completely blocked by Ara C treatment. In vSC56, β-galactosidase is expressed under the control of a synthetic E/L promoter. The overall expression is 2-fold higher compared with vSC8 and even in the presence of Ara C residual β-Gal activity was measured as previously described (Chakrabarti et al., Biotechniques, 23:1094 (1997)). At very low MOI (≦0.06 pfu/cell), no residual β-Gal activity was measured in Ara C treated cells. The virus, vSC56, was selected for further development of the methods of the invention. The experiments conducted in the current study used MOI of 0.05 pfu/cell. Each assay included virus titration spanning the desired multiplicity of infection (MOI) to ensure the integrity of the viral stock. It was determined that the control (uninhibited) values for vSC56 in HeLa should give β-Gal values between 100-300 mU/ml. Other cell lines were tested and included Vero 76 and BSC-1. The β-Gal activity measured in the lysates of HeLa cells after 16-18 hr infection was higher than in the other two cell lines. However, the ID₅₀ neutralization titers were similar (Table 2).

In order to characterize the assay, two preparations of vaccinia IgG (VIG) that were made commercially from the same source plasma were initially used. One preparation was made in 1994 for intramuscular administration (by Baxter) and the second product was made more recently by MBL for intra-venous administration (VIGIV). After identifying the dynamic range for these products, multiple neutralization assays over a period of 18 months were conducted. In each assay, virus stock was preincubated with two-fold serial dilutions of VIG or VIGIV (1:500-1:8,000) at room temperature for 60 min and the virus/antibody mixtures were added to HeLa cells. After 2 hr incubation at 37° C., the cells/virus/antibody mixtures were transferred to 96-flat bottom well plates (2×10⁵ cells/well, 6 replicates per antibody dilution), and incubated for an additional 16 hr at 37° C. in a CO₂ incubator. At that stage, cell-lysing detergent (IGEPAL CA630, 20 μl of 20% solution) was added to all wells. After thorough mixing, the plates were frozen at −70° C. After thawing, the β-Gal enzymatic assay was conducted on all control and experimental wells. For each antibody dilution, the mean #-Gal activity of 6 replicates±SD was determined and compared to the control β-Gal activity measured in the absence of inhibitors in wells infected with vSC56 at 0.05 pfu/cell. As a negative control, human albumin at the same concentrations as the VIG and VIGIV preparations was initially used. The % of control values for the 5 dilutions of VIG were plotted using the Excel program to generate straight lines by linear regression. The equation fitting each straight line was used to calculate the 50% inhibitory dilution for a given inhibitor, and this dilution was converted to ID₅₀ value in μg/ml, based on the known concentration of the VIG preparation (50 mg/ml for MBL VIGIV (FIG. 1B) (ID(50)=18 μg/ml) and 165 mg/ml for Baxter VIG).

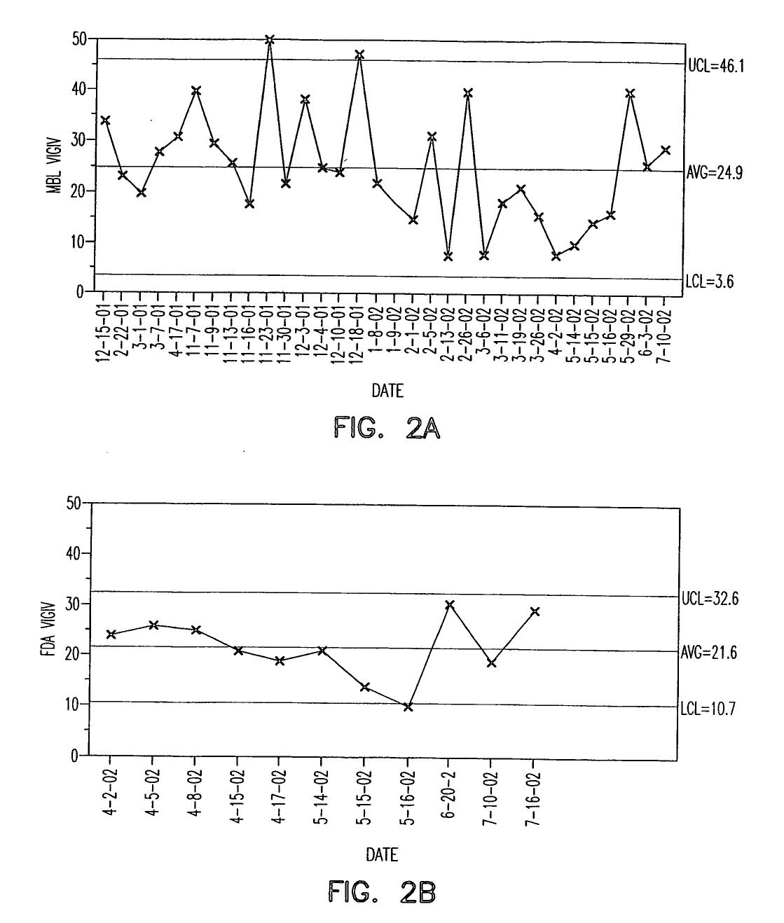

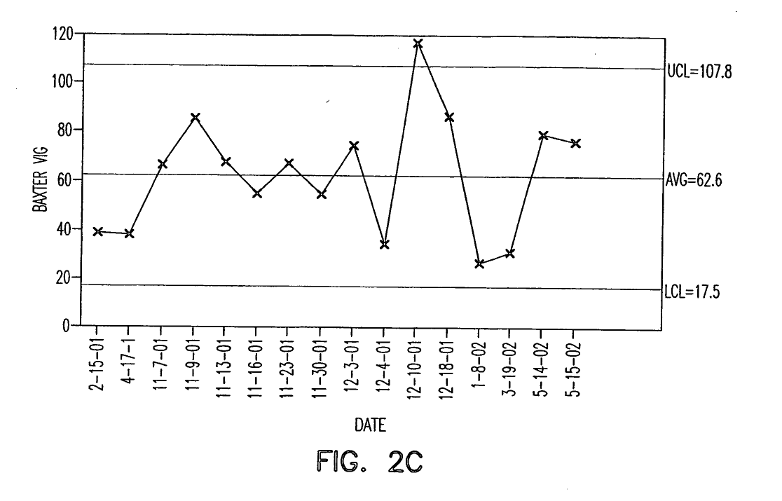

The reproducibility of the assay was established by generation of QC charts in which the mean and the upper and lower 95% confidence limit values for the VIGIV (FIG. 2A) and VIG (FIG. 2C) preparations were determined. Recently, MBL/DynPort provided CBER with a separate lot of VIGIV (lot 1) for the purpose of preparing new FDA Standard that will be available to outside laboratories in the process of establishing new vaccinia neutralization assays. The ID₅₀ value for the FDA Standard was found to be very similar to that of the previous lot of MBL VIGIV (FIG. 2B and Table 2). The FDA Standard can be freeze-thawed twice without loss of activity (Table 2).

II. Comparison of β-Gal and PRNT Vaccinia Neutralization Assays

As part of the characterization of the novel vaccinia neutralization assay, it was important to compare the results obtained using one of the inventive methods with those generated by the traditional PRNT assays. Since several groups run PRNT assays differently, the same lots of Baxter VIG and MBL VIGIV were sent to two outside laboratories. As can be seen in Table 3, the ID₅₀ values of MBL VIGIV obtained in the β-Gal neutralization assay, conducted either in HeLa cells (32 experiments) or in Vero 76 cells (4 experiments), were in very good agreement with the values obtained independently in the two other laboratories (Acambis and BioReliance), each conducting a PRNT assay with different vaccinia strains. Furthermore, both in the assay of the invention and in the PRNT assay (BioReliance) a statistically significant difference in the ID₅₀ values for Baxter VIG and MBL VIGIV (63±25 and 25±11 μg/ml, respectively) was observed (p<0.0001) (Table 4). Thus, it has been established that the newly developed β-Gal reporter-gene assay is as sensitive as the traditional PRNT assays, more rapid (24 hr), and generates 50% inhibition values similar to the PRNT assays. The BioReliance assay was recently validated and approved by the FDA for commercial use.

III. Applications of β-Gal Neutralization Assay: Screening of Human Specimens

One of the important applications of the new assay will be to evaluate the efficacy of new smallpox vaccines by measuring vaccinia neutralization titers following immunizations. To that end, 20 specimens from current individuals with a known history of smallpox vaccination were collected. Pooled serum specimens from unvaccinated children that were stored at CBER as control for other vaccine evaluations were used as negative controls. The FDA Standard was used as an internal positive control. All the specimens were heat inactivated for 30 min at 56° C. to destroy complement. The results are presented in Table 5. No viral neutralization was seen in the presence of the negative control sera at 1:20 final dilution. (specimen #21 in Table 5). The experimental results are presented in four groups. Group 1 represents individuals who received a single (known) smallpox vaccination at childhood ≧25 years ago. Group 2 includes individuals that received more than one smallpox vaccination, the most recent being ≧20 years ago. Group 3 includes individuals that were vaccinated in childhood but were boosted in the last 5 years. Group 4 included young individuals that only recently received their primary vaccination with Dryvax. The results show that the assay of the invention is sensitive enough to detect even low levels of inhibition (#5,#20). An interesting finding was that even after ≧25 years post-vaccination, some individuals maintain high titers of neutralizing antibodies. The ID₅₀ values in Ig/ml were calculated based on 10 mg/ml total I-G in human serum (column 4, in brackets). Many of these values were in the range of values obtained with MBL VIGIV. However, in some individuals in groups 1 and 2, low inhibitory titers were measured suggesting decay over the years. The majority of individuals who were boosted in the past 3 years had high neutralization titers. It was also found that naïve individuals that were primed recently and were bled 10-20 days post vaccination had varying results. ⅓ had high neutralization titer (#18) but ⅔ had very low neutralization titers (# 19 and #20). The results appear to agree with a publication describing the results of a clinical trial conducted recently, in which 60 seronegative individuals were randomized to receive undiluted, 1:10 diluted, or 1:100 diluted Dryvax vaccine (Ward et al., Proc. Natl. Acad. Sci., 92:6773 (1995)).

IV. The EGFP Reporter Gene Assay

Recombinant vaccinia expressing the gene encoding for Green Fluorescence Protein under the control of a T7 promoter as part of the VOTE expression system (Ward et al., Proc. Natl. Acad. Sci. 92:6773 (1995)), was recently constructed as a tool to develop large scale protein expression in mammalian cells (Bleckwenn, Biotechnology Progress, (2002)). Briefly, addition of the inducing agent IPTG allows production of T7 RNA polymerase that binds to the T7 promoter controlling the EGFP expression. It was of interest to determine if the new recombinant virus vNB009 can be used for vaccinia neutralization assay. In preliminary virus titration/kinetics experiments it was determined that in order to reach a dynamic range at low viral input (≦0.05 pfu/cell) infection of HeLa cells requires 44-48 hrs. Side by side tests of the FDA standard, MBL VIGIV and a new VIGIV product were then tested in the β-Gal and the EGFP reporter gene neutralization assays. As can be seen in Table 6, in both assays the ID₅₀ values for MBL and FDA Standard were very similar, while the new VIGIV product was found to be significantly better than the MBL products as judged by 2-3 fold lower ID₅₀ values. Therefore, the EGFP assay may be further developed as an alternative neutralization assay. Additional tests with human plasma specimens will be used to establish the sensitivity and reproducibility of the assay compared with the β-Gal assay.

TABLE 1
β-Gal activity in HeLa cell extracts after 18 hr infection
with serially diluted vSC8 and vSC56 recombinant viruses.

β-Gal activity^{a}

| Strain | PFU/cell | No Ara C | with Ara C |
|---|---|---|---|
| vSC8 | 1.0 | 340 ± 41 | 19 ± 2 |
| | 0.5 | 240 ± 31 | 8 ± 3 |
| | 0.25 | 175 ± 35 | 6 ± 2 |
| | 0.125 | 89 ± 10 | 3 ± 1 |
| | 0.06 | 45 ± 7 | 3 ± 1 |
| vSC56 | 1.0 | 686 ± 94 | 105 ± 28 |
| | 0.5 | 538 ± 88 | 63 ± 6 |
| | 0.25 | 442 ± 42 | 27 ± 11 |
| | 0.125 | 239 ± 16 | 15 ± 4 |
| | 0.06 | 97 ± 13 | 0 |

^{a}HeLa cells were either untreated or treated with Ara C (Sigma) at a concentration of 40 μg/ml after virus infection. Ara C blocks transcription dependent on the vaccinia late promoters (Chakrabarti et al., Biotechniques, 23: 1094 (1997)).
Data are presented as β-Gal activity in a 10 μl specimen of cell lysates (200 μl/well) after 18 hr infection. Mean ± SD for 6 replicates per MOI.

TABLE 2
50% inhibitory values of MBL VIGIV lot #2 and lot # 1
that was used to establish a new FDA Standard

| VIGIV | No. of Freeze-Thaws | No. of tests | ID₅₀ (μg/ml) |
|---|---|---|---|
| MBL Lot 2 | 1 | 32 | 25 ± 11 |
| MBL Lot 1 | 1 | 2 | 21 ± 3 |
| New FDA | 2 | 12 | 21 ± 6 |
| Standard | | | |

TABLE 3
Comparison of vaccinia neutralization assays conducted
by different laboratories with MBL VIGIV lot #2.

| | | | | ID₅₀ for MBL VIGIV |
|---|---|---|---|---|
| Laboratory | Assay | Vaccinia virus | Cell | (No. of tests) |
| Acambis | PRNT | Dryvax | Vero 76 | 16 ± 5 μg/ml (3) |
| BioReliance | PRNT | NYCBOH | Vero 76 | 24 ± 12 μg/ml (2) |
| CBER | β-Gal | vSC56 | HeLa | 25 ± 11 μg/ml (32) |
| | | | Vero 76 | 21 ± 6 μg/ml (4) |

TABLE 4
Comparison of vaccinia neutralization
with MBL VIGIV vs. Baxter VIG

| | | ID₅₀ for MBL | | ID₅₀ for Baxter | |
|---|---|---|---|---|---|
| Laboratory | Assay | VIGIV (No. tests) | | VIG (No. tests) | |
| BioReliance | PRNT | 24 ± 12 | μg/ml (2) | 58 | μg/ml (1) |
| CBER | β-Gal | 25 ± 11 | μg/ml (32) | 63 ± 25 | μg/ml (16) |

| | |
|---|---|
| Statistics | P < .0001 |
| (ANOVA)^{a} | |

^{a}The ID₅₀ values of VIGIV and VIG that were determined by repeated β-Gal assays were compared by ANOVA.

TABLE 5
50% vaccinia neutralization titers in sera collected
from individuals with known vaccination history
as determined by the β-Gal assay.

| CBER Screen | | Prime/Boost | Prime or boosts | 50% Neutralization |
|---|---|---|---|---|
| Group/individual | | (remote) | (in last 5 years) | serum titer (μg/ml) |
| Group I | 1 | 1970s | | 1:1500 (7 μg/ml) |
| | 2 | 1960s | | 1:1048 (10 μg/ml) |
| | 3 | 1960s | | 1:351 (28 μg/ml) |
| | 4 | 1960s | | 1:105 (95 μg/ml) |
| | 5 | <1960 | | 1:8 (1250 μg/ml) |
| Group II | 6 | <1960/<1970 | | 1:1500 (7 μg/ml) |
| | 7 | <1960/61; 62; | | 1:308 (32 μg/ml) |
| | | 63; 64; 65; | | |
| | | 76; 80 | | |
| | 8 | 1939/54; 57; | | 1:63 (158 μg/ml) |
| | | 59; 62; 70; | | |
| | | 76 | | |
| Group III | 9 | 1960s | 2002 | 1:1310 (8 μg/ml) |
| | 10 | 1964 | 2002 | 1:1053 (10 μg/ml) |
| | 11 | 1975/1976 | 2001 | 1:784 (13 μg/ml) |
| | 12 | 1968 | 2002 | 1:774 (13 μg/ml) |
| | 13 | 1970s | 2002 (19 dvp) | 1:497 (20 μg/ml) |
| | 14 | 1970 | 1998 | 1:344 (29 μg/ml) |
| | 15 | 1970s | 2002 | 1:212 (47 μg/ml) |
| | 16 | 1960s | 2001 | 1:188 (53 μg/ml) |
| | 17 | 1962/1972 | 2002 (19 dvp) | 1:122 (82 μg/ml) |
| Group IV | 18 | | 2002 (16 dvp) | 1:280 (36 μg/ml) |
| | 19 | | 2002 (9 dvp) | 1:36 (278 μg/ml) |
| | 20 | | 2002 (10 dpi) | 1:22 (454 μg/ml) |
| | 21 | Negative | | 0 |
| | | controls | | |
| | | (pooled 2 | | |
| | | year sera) | | |

TABLE 6
Comparison of β-Gal and EGFP reporter gene
assays for the evaluation of VIGIV products

| | | PDA Standard^{a} | MBL VIGIV^{a} | New VIGIV^{a} |
|---|---|---|---|---|
| Assay | No. of Exp. | (ID₅₀) | (ID₅₀) | (ID₅₀) |
| β-Gal^{b} | 6 | 19 ± 6 | 18 ± 5 | 8 ± 3 |
| EGFP^{b} | 4 | 10 ± 3 | 9 ± 2 | 4 ± 2 |

^{a}ID₅₀ in μg/ml were determined for each VIGIV in β-Gal or EGFP neutralization assay using vSC56 or vNB009 respectively at MOI of 0.05 pfu/cell. Results are presented as means ± S.D. for the indicated number of experiments.
^{b}Data were analyzed by ANOVA. The Tukey-Kramer HSD test was used for multi- comparison testing when the ANOVA was significant. These statistical analyses revealed significant differences (p < 0.05) between the new VIGIV and the FDA Standard or MBL VIGIV using either β-Gal or EGFP reporter gene neutralization assay.

### EXAMPLE III

### Determination of Viral Load In Vivo

Normal mice and immunodeficient mice will be infected with a vaccinia virus that encodes β-galactosidase. Tissues will be collected from the mice at different times. The tissue will be weighed, homogenized, and the β-galactosidase activity within the tissues will be determined. A β-galactosidase standard curve, as described herein, will be used to determine β-galactosidase units which will then be used to calculate the viral load contained in the collected tissues. Tissues collected will include, spleen, liver, ovaries, PBL, and lymph nodes. The viral load in all of the tissues will be calculated at all of the time points and dissemination of the vaccinia virus through the normal and immunodeficient mice will be determined.

Additional sets of normal and immunodeficient mice will be treated with vaccinia immunoglobulin G (VIG), monoclonal antibodies against vaccinia virus, or smallpox vaccines currently available. The additional mice will then be infected with a vaccinia virus that encodes β-galactosidase and processed as described above.

Another set of normal and immunodeficient mice will be infected with a vaccinia virus that encodes β-galactosidase as described above. The mice will then be treated with vaccinia immunoglobulin G (VIG), monoclonal antibodies against vaccinia virus, or smallpox vaccines currently available. The mice will then be processed as described above to determine the viral dissemination and viral load in the indicated tissues.

The data relating to viral dissemination and viral load in the various tissues will then be compared and evaluated. This evaluation will indicate therapeutic strategies that may be used to prevent, minimize, or treat infections due to smallpox. These results will then be used to design more efficient treatment strategies that can be applied to humans (Kuller et al., AIDS Res. Hum. Retroviruses, 14:1345 (1998)).

### EXAMPLE IV

### High throughput Beta-galactosidase-Based Vaccinia Neutralization Assay

I. Materials

Cells: HeLa (CCL-2 strain) cells can be obtained from ATCC, Manassas, Va. These cells are used to prepare a master cell bank (>40 vials) which allows each vial to be thawed once before use. After being thawed, the cells are maintained in Minimum Essential Medium Eagle (EMEM, Mediatech, Herndon, Va.) containing 10% fetal calf serum (Sigma-Aldrich, St. Louis, Mo.) with 2 mM L-glutamine and Earle's BSS adjusted to contain 1.5 g/L sodium bicarbonate, 0.1 mM non-essential amino acids, 1.0 mM sodium pyruvate, and antibiotics penicillin at 500 international units/ml and streptomycin at 500 μg/ml). The cells are passaged in 25 cm² flasks on a weekly basis (1:10). However, the cells were not be passaged more than 14 times. For a given experiment, cells were expanded in a 75 cm² flask, and then seeded in 5×160 cm² flasks. Only cells at >90% viability were used.

Virus Stock: vSC56 recombinant vaccinia virus was generated in the laboratory of Bernard Moss (NIAID, NIH)(Office of Technology Transfer, National Institutes of Health, 6011 Executive Boulevard, Suite 325, Rockville, Md. 20852-3804). This recombinant virus expresses the β-galactosidase gene under the control of a synthetic E/L promoter (Chalrabarti et al., BioTechniques, 23:1094 (1997)). A virus stock can be prepared by expanding HeLa cells in five 160 cm² flasks. The cells are then infected with the vSC56 vaccinia virus (1×10⁸ PFU per flask in 2 ml medium). The flasks are watched daily for CPE, until the cells contain >90% CPE. The infected cells are then detached with glass beads and transferred to 50 ml conical tubes. The tubes are spun at 1500 rpm for 5 min. Supernatants are removed and the pellets are re-suspended in PBS+0.1% BSA (2 ml/flask). The re-suspended infected cells are subjected to three freeze-thaw cycles. The cells are then frozen for 2-3 minutes in a dry ice/alcohol bath (with swirling). The cells are thawed for 2-3 minutes in a 37° C. water bath (with swirling). The freeze-thawed infected cells are aliquoted in small volumes (<0.5 ml/vial) and the vials containing the virus stocks are frozen at −70° C.

Trypsin treatment of virus stocks: A 1:10 dilution of a 10× stock of trypsin can be prepared (2.5% w/v in Hanks Balanced Salt Solution from Mediatech) in calcium-free and magnesium-free PBS. Equal volumes of virus stock and diluted trypsin are mixed in a microtube and incubated in a 37° C. water-bath for 30 minutes prior to preparing the appropriate virus dilutions for the assay to break up virus clumps. Alternatively, the virus stocks can be sonicated to break-up clumps.

Virus titration: One vial of cells are thawed and serially diluted after mild treatment with trypsin to disrupt virus aggregates. Virus stock is serially diluted in EMEM medium containing 10% FCS (10⁻⁴, 10⁻⁵, 10⁻⁶, 10⁻⁷) and added to BSC-1 cells. Each virus dilution is added to two 25 cm² flasks of BSC-1 cells. On the second day, the supernates are removed from the flasks and 0.1% Crystal violet solution in 20% ethanol is added to the flasks. The plaques are then counted that the virus titer is calculated. This procedure is repeated with another working viral seed. The viral titers obtained in two independent titrations should be within 0.5 log. The viral titers of the first and second viral seeds are averaged. Typical viral titers are between 10⁸-10⁹ plaque forming units/ml.

II. Neutralization Assay Set Up

Each assay includes a titration of vSC56 at MOI of 0.125, 0.06, 0.03 PFU/cell to assure linearity of viral dilutions and to obtain control values for the assay. Most assays are run at a MOI of 0.06 or 0.125 PFU/cell. β-gal values in the absence of inhibitors are typically between 400-800 mU/ml. Viral antibodies for neutralization are prepared by heat-treating all serum or plasma specimens for 30 min at 56° C. in a water-bath to inactivate complement.

FDA VIGIV standard (positive control) and test articles (immune sera, new VIG) and negative controls (sera from unvaccinated individuals and albumin) are serially diluted. Four or five two-fold or three-fold dilutions across the dynamic range are used for each test article. The dynamic range was determined in a preliminary experiment.

All test articles and virus is prepared at two-fold final concentrations. All antibody (or serum) dilutions are prepared in EMEM supplemented with 10% fetal calf serum. For each antibody dilution, 250 μl of diluted specimen is combined with (at two-fold final concentration) 100 μl of virus (at predetermined optimal MOI) and 150 μl of EMEM in microtubes (Sarstedt). The combination is incubated at 37° C. for 1 hour with intermittent mixing.

During the incubation time, HeLa cells (in EMEM+10% FCS) are dispensed in a 96 well round bottom plate (1×10⁵ cells per well in 50 μl, 4-5 replicates per group). After a 1 hour incubation period, 50 μl of the virus-antibody mixtures are added to the HeLa cell-containing wells.

For the virus only titration, 100 μl of virus (3 different MOI spanning the optimal MOI) is combined with 400 μl EMEM/10% fetal calf serum, and incubated for 1 hour at 37° C.

FDA Standard (50 mg/ml) and other VIG preparations are tested at two-fold dilutions between 1:500->1:8000 final dilution.

Human specimens are tested at three-fold dilutions of either 1:20->1:540 (for primary vaccination with Dryvax or prime/boost vaccination with attenuated vaccine strains), or at 1:60->1620 for re-vaccination with Dryvax. Negative control sera are tested at two-fold dilutions of 1:20->1:160.

Virus titration plates and virus neutralization plates are incubated for an additional 16-18 hours at 37° C. in a humidified CO² incubator.

On the next day, 100 μl of a 4% solution of the detergent IGEPAL CA630 (Sigma-Aldrich) in EMEM/10% FCS is added to each well to lyse the HeLa cells. The plates are then incubated at 37° C. for 30 min. At this stage, the plates can be stored at −70° C. for up to a month.

III. β-Galactosidase Assay

Reagents

1M Na₂CO₃; 0.1M sodium phosphate (pH 7.0); 1M KCL; 1M MgSO₄; β-mercaptoethanol (Sigma)

Z buffer: 98.55 ml 0.1M sodium phosphate (100 mM); 1 ml 1M KCL (10 mM); 0.1 ml 1M MgSO₄ (1 mM); 0.35 ml β-mercaptoethanol (Sigma)

CPRG Substrate: 4 mg/ml CPRG (Roche Diagnostics) in 0.1M sodium phosphate (pH 7.0)

β-Galactosidase standard: Recombinant β-Galactosidase enzyme (liquid form, at 1500 units/ml (Roche Diagnostics)) distributed to small vials and stored at 4° C.

Working solution of β-Gal (total volume: 2 ml): Glycerol (1 ml); BSA (Gibco/BRL) (8 μl of 50 mg/ml dissolved in PBS (equal to 400 μg)); PBS (972 μl); β-Galactosidase stock enzyme (1500 units/ml) (20 μl); Mix thoroughly and distribute into 8 μl/vial aliquots. Store at −20° C. The final β-Gal concentration in the working solution is 15 units/ml=15,000 mU/ml.

Assay Set-Up

The plates are thawed completely and keep at 4° C. for the duration of the assay set-up. The plates can be re-frozen at −70° C. Mix each well thoroughly and transfer 20 μl into wells containing 180 μl Z Buffer (1:10 dilution).

Set up a 96 well Immulon-2 plate (Dynatech).

Prepare a β-Gal standard by serially diluting the β-Gal working solution (15,000 mU/ml) in Epindorf tubes containing Z buffer+PBS at a 4:1 ratio on ice. Typically the β-Gal solution is serially diluted by first preparing a 1:100 dilution (150 mU/ml), followed by 2-fold dilutions. Prepare enough for several plates.

In each Immulon plate, dispense the β-Gal standards in duplicate in the first 7 wells in columns 1 and 2, starting at 37.5 mU/ml and going down to 0.6 mU/ml.

The last 2 wells in columns 1+2 are the Blank wells (to blank the machine), containing 20 μl of PBS+80 μl Z buffer

Experimental Set-Up:

In each Immulon plate, add 180 μl of Z buffer to all wells in columns 3 thru 12. Transfer 20 μl from each replicate well in the original set-up plate (after thawing and mixing of wells) into the Immulon plate well containing 180 μl Z buffer to form a 1:10 dilution, and mix well. For each serum dilution (or virus only controls), there should be a minimum of 4 replicates. Add 20 μl of the CPRG substrate to all wells (prepared just before use). Incubate in the dark at room temperature for 30 min. After a 30 minute incubation, add 50 μl of 1M Na₂CO₃ to stop the reaction. Read the plates in an ELISA reader at 575 nm wavelength. Blank on the “Blank wells”. Save data.

Calculations

Use Microsoft Excel to generate a linear standard curve by plotting the OD₅₇₅ values vs. enzyme concentrations of the β-galactosidase enzyme at 37.5, 18.75, 9.38, 4.69, 2.34, 1.17, 0.59, and 0 mU/ml. These enzyme concentrations should be in the dynamic range of the machine at 575 nm (OD₅₇₅). If r² of the standard curve is <0.99, the standard curve (and the assay) is unacceptable. Use the equation (y=bx+a) of the linear standard curve and the initial sample dilution factor (1:100) to convert from OD₅₇₅ values of each sample to β-Gal activity in mU/ml. Calculate the mean±standard deviation (SD) for each group of 4-5 replicates. SD should not exceed 15% of means.

Using the β-Gal activity in the virus only plate (at 0.06 or 0.03 pfu/cell) as a 100% value (uninhibited control), calculate for each experimental group “% of control” value.

Use Microsoft Excel to plot the logs of sample dilutions (column A) vs. the “% of control” values (column B) for each test specimen or VIG Standard. The r² of the “% of Control” curve should be >0.9 (i.e., good linearity). Use the equation of the linear curves to calculate the 50% inhibitory dilution (ID₅₀) (same as 50% neutralizing titer) for each test specimen. For FDA Standard only, divide the concentration of the stock solution (50,000 μg/ml) by the 50% inhibitory dilution. This will give the ID₅₀ in μg/ml.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method comprising:
incubating a mixture comprising at least one cell, a labeled invasin that encodes a detectable label, and a candidate agent under conditions wherein the labeled invasin can invade the cell; and
detecting the detectable label within the cell, wherein an increase or decrease of detectable label in the cell due to the candidate agent indicates that the candidate agent modulates invasion of the cell by the invasin.

2. The method of claim 1, wherein the candidate agent increases invasion of the cell by the labeled invasin.

3. The method of claim 1, wherein the candidate agent decreases invasion of the cell by the labeled invasin.

4. The method of claim 1, wherein the labeled invasin is a virus.

5. The method of claim 4, wherein the virus is an enveloped virus.

6. The method of claim 5, wherein the enveloped virus is selected from the group consisting of herpesvirus, orthomyxovirus, retrovirus, and influenza viruses.

7. The method of claim 5, wherein the enveloped virus is vaccinia virus.

8. The method of claim 5, wherein the enveloped virus is a smallpox virus.

9. The method of claim 4, wherein the virus is a non-enveloped virus.

10. The method of claim 9, wherein the non-enveloped virus is an adenovirus.

11. The method of claim 1, wherein the labeled invasin is a bacterium.

12. The method of claim 1, wherein the detectable label is a fluorescent protein.

13. The method of claim 1, wherein the detectable label is an enzyme.

14. The method of claim 1, wherein the candidate agent is a peptide, a compound, a monoclonal antibody, a polyclonal antibody, an altered antibody, or an enzyme.

15. The method of claim 1, wherein the candidate agent associates with the labeled invasin.

16. The method of claim 1, wherein the candidate agent associates with the cell.

17. The method of claim 1, wherein the cell is a mammalian cell.

18. The method of claim 17, wherein the cell is a human cell.

19. The method of claim 16, wherein the candidate agent associates with a receptor on the cell.

20. The method of claim 19, wherein the receptor is selected from the group consisting of an Fc receptor, heparin sulfate receptor, vitronectin receptor, Vcam-1 receptor, hemaglutinin receptor, Pvr receptor, Icam-1 receptor, decay-accelerating protein (CD55) receptor, Car (coxsackievirus-adenovirus) receptor, integrin receptor, sialic acid receptor, HAVCr-1 receptor, low-density lipoprotein receptor, BGP (biliary glycoprotein) receptor, aminopeptidease N receptor, MHC class-1 receptor, laminin receptor, nicotinic acetylcholine receptor, CD56 receptor, nerve growth factor receptor, CD46 receptor, asialoglycoprotein receptor Gp-2, alpha-dystroglycan receptor, galactosylceramide receptor, Cxcr4 receptor, Glvr1 receptor, Ram-1 receptor, Cat receptor, Tva receptor, BLVRcp1 receptor, MHC class-2 receptor, and a complement receptor.

21. The method of claim 18, wherein the cell is selected from the group consisting of a lymphoid cell, a pulmonary cell, and an intestinal cell.

22. An agent identified according to the method of claim 1.

23. A method comprising:
incubating a mixture comprising a cell, a specimen suspected of containing at least one antibody, and a labeled invasin encoding a detectable label under conditions wherein the labeled invasin can invade the cell; and
detecting the detectable label within the cell, wherein a decrease in the detectable label in the cell due to the specimen indicates that the specimen contains an antibody that decreases invasion of the cell by the invasin.

24. The method of claim 23, wherein the specimen is an antibody preparation.

25. The method of claim 23, wherein the specimen is an immunogenic specimen.

26. The method of claim 25, wherein the immunogenic specimen is serum.

27. The method of claim 26, wherein the serum is from a human.

28. The method of claim 23, wherein the specimen is supernatant from antibody secreting cells.

29. The method of claim 28, wherein the antibody secreting cells are hybridomas.

30. The method of claim 23, wherein the specimen is an antibody preparation.

31. The method of claim 23, wherein the antibody binds to the invasin.

32. The method of claim 23, wherein the antibody binds to the cell.

33. The method of claim 32, wherein the antibody binds to a receptor on the cell.

34. The method of claim 33, wherein the receptor is selected from the group consisting of an Fc receptor, heparin sulfate receptor, vitronectin receptor, Vcam-1 receptor, hemaglutinin receptor, Pvr receptor, Icam-1 receptor, decay-accelerating protein (CD55) receptor, Car (coxsackievirus-adenovirus) receptor, integrin receptor, sialic acid receptor, HAVCr-1 receptor, low-density lipoprotein receptor, BGP (biliary glycoprotien) receptor, aminopeptidease N receptor, MHC class-1 receptor, laminin receptor, nicotinic acetylcholine receptor, CD56 receptor, nerve growth factor receptor, CD46 receptor, asialoglycoprotein receptor Gp-2, alpha-dystroglycan receptor, galactosylceramide receptor, Cxcr4 receptor, Glvr1 receptor, Ram-1 receptor, Cat receptor, Tva receptor, BLVRcp1 receptor, MHC class-2 receptor, and a complement receptor.

35. The method of claim 23, wherein the invasin is a virus.

36. The method of claim 35, wherein the virus is an enveloped virus.

37. The method of claim 36, wherein the enveloped virus is selected from the group consisting of herpesvirus, orthomyxovirus, retrovirus, and influenza viruses.

38. The method of claim 35, wherein the virus is smallpox.

39. The method of claim 35, wherein the virus is a non-enveloped virus.

40. The method of claim 39, wherein the non-enveloped virus is an adenovirus.

41. The method of claim 23, wherein the invasin is a bacterium.

42. The method of claim 23, wherein the antibodies neutralize invasion of the cell by the invasin.

43. The method of claim 23, further comprising titering the antibodies in the specimen.

44. The method of claim 43, wherein the specimen is vaccinia immunoglobulin G (VIG).

45. The method of claim 23, wherein a concentration of antibodies necessary to neutralize invasion of a cell by an invasin is determined.

46. A method comprising:
incubating a mixture comprising, an immunogenic specimen obtained from a human, a vaccinia virus that encodes a detectable label, and at least one cell under conditions wherein the vaccinia virus can infect the cell; and
detecting the detectable label in the cell, wherein a decrease in the detectable label within the cell due to the immunogenic specimen indicates that the human was exposed to smallpox.

47. The method of claim 46, wherein the immunogenic specimen is blood.

48. The method of claim 46, wherein the immunogenic specimen is serum.

49. The method of claim 46, wherein the detectable label is an enzyme.

50. The method of claim 49, wherein the enzyme is O-galactosidase.

51. The method of claim 46, wherein the label is a fluorescent protein.

52. The method of claim 46, further comprising tittering antibodies present in the immunogenic specimen that reduce invasion of the cell by the invasin.

53. A method comprising:
incubating a mixture comprising, an immunogenic specimen obtained from a human that was vaccinated with a smallpox vaccine, a vaccinia virus that expresses a detectable label, and at least one cell under conditions wherein the vaccinia virus can infect the cell; and
detecting the detectable label within the cell, wherein a decrease in the detectable label in the cell due to the immunogenic specimen indicates that the smallpox vaccine caused production of antibodies in the human that bind to the vaccinia virus.

54. The method of claim 53, wherein the immunogenic specimen is blood.

55. The method of claim 53, wherein the immunogenic specimen is serum.

56. The method of claim 53, wherein the detectable label is an enzyme.

57. The method of claim 56, wherein the detectable label is β-galactosidase.

58. The method of claim 53, wherein the detectable label is a fluorescent protein.

59. A method comprising:
incubating a mixture comprising, an invasin that encodes a detectable label and has a preselected antigen on its surface, a specimen suspected of containing an antibody that binds to the preselected antigen, and at least one cell, under conditions wherein the invasin can invade the cell; and
detecting the detectable label within the cell, wherein a decrease in the detectable label in the cell due to the specimen indicates that the specimin contains an antibody that binds to the preselected antigen.

60. The method of claim 59, wherein the invasin is an enveloped virus that was packaged in a packaging cell that displays the preselected antigen on its surface.

61. The method of claim 60, wherein the packaging cell is a recombinant cell that expresses an exogenous nucleic acid segment encoding the preselected antigen.

62. The method of claim 61, wherein the preselected antigen is a fusion protein having a predetermined peptide fused to a membrane localization signal.

63. The method of claim 62, wherein the predetermined peptide is used as a vaccine.

64. The method of claim 63, wherein the predetermined peptide is human immunodeficiency virus gp120.

65. The method of claim 59, wherein the invasin is a recombinant enveloped virus that expresses the preselected antigen within a packaging cell, wherein the preselected antigen is displayed on the surface of the packaging cell and is also displayed on the enveloped virus packaged by the packaging cell.

66. The method of claim 65, wherein the preselected antigen is a fusion protein having a predetermined peptide fused to a membrane localization signal.

67. The method of claim 66, wherein the predetermined peptide is a subunit vaccine.

68. The method of claim 66, wherein the predetermined peptide is human immunodeficiency virus gp120.

69. The method of claim 59, further comprising titering the antibody in the specimen.

70. A method comprising:
incubating a mixture comprising an antibody raised against a cell membrane preparation, an invasin that encodes a detectable label, and at least one cell; and
detecting the detectable label within the cell, wherein a decrease in the detectable label in the cell due to the antibody indicates that the antibody binds to a receptor used by the invasin to invade the cell.

71. The method of claim 70, wherein the invasin is a virus.

72. The method of claim 71, wherein the virus is an enveloped virus.

73. The method of claim 71, wherein the virus is smallpox.

74. The method of claim 71, wherein the virus is a non-enveloped virus.

75. The method of claim 70, wherein the invasin is a bacteria.

76. The method of claim 70, further comprising isolating the cellular receptor.

77. The method of claim 76, wherein a virus overlay protein blot assay, gene-transfer, or cDNA-transfer is used to isolate the cellular receptor.

78. The method of claim 76, wherein the cellular receptor is immunoseparated.

79. The method of claim 76, further comprising determining the amino acid sequence of the cellular receptor.

80. The method of claim 70, wherein the cell is transformed with an exogenous nucleic acid segment encoding the receptor used by the invasin to invade the cell.

81. The method of claim 80, wherein the cell did not express the receptor used by the invasin to invade the cell prior to being transformed with the exogenous nucleic acid segment.

82. The method of claim 70, wherein the antibody is a polyclonal antibody.

83. The method of claim 70, wherein the antibody is a monoclonal antibody.

84. A method comprising:
incubating a mixture comprising, an invasin that encodes a detectable label, a candidate agent, a subneutralizing concentration of antibodies that bind to the invasin, and at least one cell under conditions wherein the invasin can invade the cell; and
detecting the detectable label within the cell, wherein an increase or decrease in the detectable label in the cell due to the candidate agent indicates that the candidate agent modulates antibody-mediated infection of the cell.

85. The method of claim 84, wherein the invasin is a virus.

86. The method of claim 85, wherein the virus is an enveloped virus.

87. The method of claim 85, wherein the enveloped virus is smallpox.

88. The method of claim 85, wherein the virus is a non-enveloped virus.

89. The method of claim 84, wherein the invasin is a bacterium.

90. The method of claim 84, wherein the cell is a transformed cell that contains an exogenous DNA encoding a receptor used by the invasin to invade the cell.

91. The method of claim 90, wherein the transformed cell did not express the receptor used by the invasin to invade the cell prior to being transformed with the exogenous DNA.

92. The method of claim 90, wherein the exogenous DNA encodes an Fc receptor.

93. A method comprising:
incubating a mixture comprising a cell monolayer having a first side and a second side, wherein an invasin that encodes a detectable label is contacted with the cell monolayer on the first side, and an antibody is contacted with the cell monolayer on the second side; and
detecting the detectable label on the second side of the cell monolayer following incubation, wherein an increased or decreased amount of the detectable label on the second side of the cell monolayer due to the antibody indicates that the antibody mediates transport of the invasin across the cell monolayer.

94. The method of claim 93, wherein the antibody increases transport of the invasin across the cell monolayer.

95. The method of claim 93, wherein the antibody decreases transport of the invasin across the cell monolayer.

96. The method of claim 93, wherein the invasin is a virus.

97. The method of claim 96, wherein the virus is an enveloped virus.

98. The method of claim 96, wherein the virus is a non-enveloped virus.

99. The method of claim 93, wherein the invasin is a bacterium.

100. The method of claim 99, wherein the bacterium is selected from the group consisting of Shigella, Listeria, Salmonella, Tuberculosis, B. abortis, Chlamydia, and Leprosy.

101. The method of claim 93, wherein the mixture further comprises a candidate agent.

102. The method of claim 101, further comprising detecting the detectable label on the second side of the cell monolayer following incubation, wherein an increased or decreased amount of the detectable label on the second side of the cell monolayer due to the candidate agent indicates that the candidate agent mediates transport of the invasin across the cell monolayer.

103. The method of claim 93, wherein the cell monolayer comprises intestinal epithelium cells.

104. A method to assay invasin load in vivo comprising:
infecting an organism with an invasin that encodes a detectable label to produce an infected organism; and
detecting the detectable label within a specimen obtained from the infected organism.

105. The method of claim 104, wherein the organism is an avian, a mammal, or a reptile.

106. The method of claim 104, wherein the mammal is a rat, mouse, horse, dog, cat, ferret, mink, weasel, beaver, muskrat, rabbit, or cow.

107. The method of claim 104, wherein the organism is immunodeficient.

108. The method of claim 107, wherein the organism is an avian, a mammal, or a reptile.

109. The method of claim 108, wherein the mammal is a rat, mouse, horse, dog, cat, ferret, mink, weasel, beaver, muskrat, rabbit, cow, or human.

110. The method of claim 104, wherein the invasin is a bacterium.

111. The method of claim 104, wherein the invasin is a virus.

112. The method of claim 111, wherein the virus is an enveloped virus.

113. The method of claim 112, wherein the enveloped virus is selected from the group consisting of alphavirus, rhabdovirus, orthomyxovirus, retrovirus, and herpesvirus.

114. The method of claim 112, wherein the enveloped virus is vaccina virus.

115. The method of claim 112, wherein the virus is a non-enveloped virus.

116. The method of claim 104, wherein the specimen is liver, spleen, lymph nodes, ovaries, blood, or serum.

117. The method of claim 104, wherein the detectable label is an enzyme.

118. The method of claim 117, wherein the enzyme is β-galactosidase.

119. The method of claim 117, wherein the detectable label is a fluorescent protein.

120. The method of claim 104, further comprising determining the kinetics of viral dissemination.

121. The method of claim 120, further comprising determining if administration of a vaccine, an antibody preparation, a monoclonal antibody, an enzyme, a peptide, a compound, a pharmaceutical composition, or any combination thereof, before the organism is infected with the invasin modulates viral dissemination.

122. The method of claim 120, further comprising determining if administration of a vaccine, an antibody preparation, a monoclonal antibody, an enzyme, a peptide, a compound, a pharmaceutical composition, or any combination thereof, after the organism is infected with the invasin modulates viral dissemination.

123. A kit comprising packaging material, an invasin that encodes a detectable label, and a cell that the invasin can invade.

124. The kit of claim 123, wherein the invasin is a vaccinia virus.

125. The kit of claim 124, wherein the detectable label is an enzyme.

126. The kit of claim 125, wherein the enzyme is β-galactosidase.

127. The kit of claim 123, wherein the detectable label is a fluorescent protein.

128. The kit of claim 123, wherein the cell is a HeLa cell.

129. A kit comprising packaging material and an invasin that encodes a detectable label.
